(19) United States
(12) Patent Application Publication
(10) Pub. No.: US 2009/0069230 A1
(43) Pub. Date: Mar. 12, 2009

(54) GDNF-DERIVED PEPTIDES

(75) Inventors: Srinivas N. Pentyala, South Setauket, NY (US); John D. Glass, Shoreham, NY (US)

Correspondence Address: FISH & RICHARDSON P.C., P.O. BOX 1022, MINNEAPOLIS, MN, 55440-1022, US

(73) Assignee: The Research Foundation of State University of New York, Stony Brook, NY (US)

(21) Appl. No.: 12/030,172

(22) Filed: Feb. 12, 2008

Related U.S. Application Data
(60) Provisional application No. 60/889,467, filed on Feb. 12, 2007.

(51) Int. Cl. A61K 38/22; C07K 14/575; C07H 21/04; C12N 15/64; C12N 5/10; A61P 25/28

(52) U.S. Cl. 514/12; 530/324; 536/23.51; 435/320.1; 435/366

(57) ABSTRACT

Compositions that include peptides derived from glial-derived neurotrophic factor (GDNF) (e.g., substantially pure polypeptides comprising a fragment of a GDNF precursor protein) and biologically active variants thereof are provided. The compositions can include one or more types of peptides and can include other substances (e.g., pharmaceutically acceptable carriers or diluents or liposomes). Also provided are methods for using the compositions for treatment of neurological disorders (e.g., motor system disorders and sensory system disorders).

### CROSS-REFERENCE TO PRIORITY APPLICATION

This application claims priority to U.S. Provisional Application No. 60/889,467, filed Feb. 12, 2007. The application to which the present application claims benefit is herein incorporated by reference in its entirety. The work below was funded in part by a grant from Stony Brook University/Brookhaven National Laboratories, Seed Grant Number: 1050534-37298 (2005). The U.S. government therefore has certain rights in the invention.

### TECHNICAL FIELD

This invention relates to treating neurological disorders, and more particularly to methods of making and using peptides derived from glial-derived neurotrophic factor (GDNF) to treat such disorders.

### BACKGROUND

Neurological disorders encompass a wide range of diseases and conditions that affect the brain and other parts of the nervous system. Neurological disorders remain a major cause of mortality and morbidity and the extreme pain, excessive movement, memory loss, and muscle degeneration that often accompanies many such disorders can affect every aspect of a person's daily life. For many patients with neurological disorders, the available remedies consist primarily of palliative care rather than curative treatments.

Neuronal damage and degeneration play major causative roles in many diverse neurological disorders, including Parkinson's disease, chronic neurodegenerative conditions, acute stroke, developmental disorders, and multiple sclerosis.

### SUMMARY

The present invention is based, in part, on our discovery of compositions and methods that can be used to treat neurological disorders. Neurological disorders encompassed by the invention can include motor system disorders (e.g., Parkinson's disease, ataxia, and dyskinesia) as well as neurologic syndromes (e.g., attention deficit hyperactivity disorder (ADHD)) and sensory disorders (e.g., hearing loss or impairment).

The compositions include polypeptides derived from a GDNF protein, which may be either a GDNF precursor protein or a mature GDNF protein (e.g., a human GDNF) and/or biologically active variants thereof. A polypeptide that has a sequence that is identical to a portion of a GDNF sequence and that functions (e.g., for one or more of the purposes described herein) is a fragment of a GDNF precursor protein. Of course, the GNDF precursor protein includes the mature GDNF sequence and one or more of the polypeptides described herein may lie partially or wholly within the mature GDNF sequence. A polypeptide that has a sequence that differs to a certain limited extent from a sequence that is found in a naturally occurring GDNF and that retains the ability to function (e.g., retains sufficient activity to be used for one or more of the purposes described herein) is a biologically active variant of a GDNF precursor protein. We tend to use the terms “GDNF”, “GDNF protein” and “GDNF precursor protein” to refer to full-length, naturally-occurring GDNF proteins, and we tend to use the terms “polypeptide” and “peptide” when referring to fragments thereof (i.e., to fragments of GDNF precursor proteins) and biologically active variants thereof. Because the polypeptides or peptides can have a sequence that is identical to a sequence found in GDNF, the polypeptides or peptides are derived from fragments of a GDNF precursor protein.

The compositions including fragments of a GDNF precursor protein and/or biologically active variants thereof can be formulated in various ways and can include pharmaceutically acceptable carriers. For ease of reading, we do not repeat the phrase “and/or biologically active variants thereof” after every reference to a fragment of a GDNF precursor protein. It is to be understood that where a fragment of a GDNF precursor protein is useful, a variant of the polypeptide that has comparable biological activity (e.g., sufficient activity to be used for one or more of the purposes described herein (e.g., for the purpose for which one would have used a fragment of a GDNF precursor protein)) is also useful. The fragment of a GDNF precursor protein can be a fragment of any isoform of GDNF, for example, isoform 1 or isoform 2.

Accordingly, the invention features physiologically acceptable compositions and concentrated stocks of fragments of a GDNF precursor protein and methods by which those fragments can be prepared and formulated for administration to a patient diagnosed as having, for example, a neurological disorder. While the sequences of the present polypeptides can vary, useful polypeptides can include not more than 92 amino acid residues and/or can exclude amino acid residues 118-211 of SEQ ID NO:8. The polypeptides can include or consist of an amino acid sequence of a GDNF that is naturally expressed in a mammalian cell (e.g., a human cell). A biologically active variant can include, for example, an amino acid sequence that differs from a wildtype fragment of a GDNF precursor protein by virtue of containing one or more conservative amino acid substitutions, with the proviso that at least 50% of the amino acid residues of the variant are identical to residues in the corresponding wildtype fragment of a GDNF precursor protein. Biologically active variants can also include amino acid sequences that differ from a wildtype fragment of a GDNF precursor protein by virtue of non-conservative amino acid substitutions, additions, and/or deletions. These variants are described in more detail below.

More specifically, the fragments of a GDNF precursor protein can have, or can include, a sequence of amino acid residues conforming to the amino acid sequence of Formula I:

(SEQ ID NO:9)

Xaa₁-Pro-Xaa₃-Pro-Xaa₅-Xaa₆-Xaa₇-Xaa₈ (I).

Within this formula, Xaa₁ is Phe, Trp, or Tyr; Xaa₃ is Leu, Ala, Ile, or Val; Xaa₅ is Ala, Leu, Ile, or Val; Xaa₆ is Gly, is any amino acid residue of the D configuration or is absent; Xaa₇ is Lys, Arg, or His or is absent; and Xaa₈ is Arg, Lys, or His or is absent. Xaa represents an amino acid, which we may also refer to as an amino acid residue. The subscripts (here, the subscripts 1-8) represent the positions of each amino acid in the peptide sequence. Thus, Xaa₁ represents the first amino acid residue in a fragment of a GDNF precursor protein. The invention encompasses peptides (e.g., substantially pure peptides) that consist of the referenced sequences (e.g., a peptide conforming to any of the formulas described herein) or that include the referenced sequences and additional sequence as described herein. The invention excludes full length, naturally-occurring GDNF proteins.

In specific embodiments, the fragments of a GDNF precursor protein can conform to the following specifications. The peptide can have a sequence represented by:

(1) Phe-Pro-Xaa₃-Pro-Xaa₅-Xaa₆-Xaa₇-Xaa₈ (SEQ ID NO:10), where Xaa₃ and Xaa₅-Xaa₆-Xaa₇-Xaa₈ are, independently, as described above (e.g., Phe-Pro-Leu-Pro-Ala-Gly-Lys-Arg (SEQ ID NO:1); (2) Xaa₁-Pro-Leu-Pro-Xaa₅-Xaa₆-Xaa₇-Xaa₈ (SEQ ID NO:11), where Xaa₁, and Xaa₅-Xaa₆-Xaa₇-Xaa₈ are, independently, as described above; (3) Phe-Pro-Leu-Pro-Xaa₅-Xaa₆-Xaa₇-Xaa₈ (SEQ ID NO:12), where Xaa₅-Xaa₆-Xaa₇-Xaa₈ are, independently, as described above; (4) Xaa₁-Pro-Xaa₃-Pro-Ala-Xaa₆-Xaa₇-Xaa₈ (SEQ ID NO:13), where Xaa₁, Xaa₃ and Xaa₆-Xaa₇-Xaa₈ are, independently, as described above; (5) Phe-Pro-Xaa₃-Pro-Ala-Xaa₆-Xaa₇-Xaa₈ (SEQ ID NO:14), where Xaa₃ and Xaa₆-Xaa₇-Xaa₈ are, independently, as described above; (6) Phe-Pro-Leu-Pro-Ala-Xaa₆-Xaa₇-Xaa₈ (SEQ ID NO:15), where Xaa₆, Xaa₇, and Xaa₈ are, independently, as described above; (7) Xaa₁-Pro-Xaa₃-Pro-Xaa₅-Gly-Xaa₇-Xaa₈-(SEQ ID NO:16), where Xaa₁, Xaa₃, Xaa₅, and Xaa₇-Xaa₈ are, independently, as described above; (8) Phe-Pro-Xaa₃-Pro-Xaa₅-Gly-Xaa₇-Xaa₈ (SEQ ID NO:17), where Xaa₃, Xaa₅, and Xaa₇-Xaa₈ are, independently, as described above; (9) Phe-Pro-Leu-Pro-Xaa₅-Gly-Xaa₇-Xaa₈ (SEQ ID NO:18), where Xaa₅, and Xaa₇-Xaa₈ are, independently, as described above; (10) Phe-Pro-Leu-Pro-Ala-Gly-Xaa₇-Xaa₈ (SEQ ID NO:19), where Xaa₇ and Xaa₈ are, independently, as described above; (11) Xaa₁-Pro-Xaa₃-Pro-Xaa₅-Xaa₆-Lys-Xaa₈ (SEQ ID NO:20), where Xaa₁, Xaa₃, Xaa₅, Xaa₆ and Xaa₈ are, independently, as described above; (12) Phe-Pro-Xaa₃-Pro-Xaa₅-Xaa₆-Lys-Xaa₈ (SEQ ID NO:21), where Xaa₃, Xaa₅, Xaa₆ and Xaa₈ are, independently, as described above; (13) Phe-Pro-Leu-Pro-Xaa₅-Xaa₆-Lys-Xaa₈ (SEQ ID NO:22), where Xaa₅, Xaa₆ and Xaa₈ are, independently, as described above; (14) Phe-Pro-Leu-Pro-Ala-Xaa₆-Lys-Xaa₈ (SEQ ID NO:23), where Xaa₆ and Xaa₈ are, independently, as described above; (15) Phe-Pro-Leu-Pro-Ala-Gly-Lys-Xaa₈ (SEQ ID NO:24), where Xaa₈ is as described above; (16) Xaa₁-Pro-Xaa₃-Pro-Xaa₅-Xaa₆-Xaa₇-Arg (SEQ ID NO:25) where Xaa₁, Xaa₃, Xaa₅, Xaa₆ and Xaa₇ are, independently, as described above; (17) Phe-Pro-Xaa₃-Pro-Xaa₅-Xaa₆-Xaa₇-Arg (SEQ ID NO:26) where Xaa₃, Xaa₅, Xaa₆ and Xaa₇ are, independently, as described above; (18) Phe-Pro-Leu-Pro-Xaa₅-Xaa₆-Xaa₇-Arg (SEQ ID NO:27) where Xaa₅, Xaa₆ and Xaa₇ are, independently, as described above; (19) Phe-Pro-Leu-Pro-Ala-Xaa₆-Xaa₇-Arg (SEQ ID NO:28), where Xaa₆ and Xaa₇ are, independently, as described above; (20) Phe-Pro-Leu-Pro-Ala-Gly-Xaa₇-Arg (SEQ ID NO:29), where Xaa₇ is as described above; and so forth. Given the present formulas, the present provisions, and examples such as these, one of ordinary skill in the art is well able to select various substituted amino acid residues from among those permitted and to make and use peptides conforming to the present formulas and variants thereof.

In a more specific embodiment, the fragment of a GDNF precursor protein can be a fragment or portion of a GDNF precursor protein conforming to Formula I, where Xaa₁ is Phe, Xaa₃ is Leu, Xaa₅ is Ala, Xaa₆ is Gly, Xaa₇ is Lys and Xaa₈ is Arg (i.e., Phe-Pro-Leu-Pro-Ala-Gly-Lys-Arg (SEQ ID NO:1)). At least one (e.g., one, two, or three) of the amino acid residues represented by Formula I can be absent. For example, Xaa₆, Xaa₇, and/or Xaa₈ can be absent.

In another embodiment, the fragments of a GDNF precursor protein or the biologically active variants can have, or can include, a sequence of amino acid residues conforming to the amino acid sequence of Formula II:

(SEQ ID NO:30)

Pro-Pro-Xaa₃-Xaa₄-Pro-Xaa₆-Xaa₇-Xaa₈-Xaa₉-Xaa₁₀-
Xaa₁₁-Xaa₁₂-Xaa₁₃-Xaa₁₄ (II).

Within this Formula, Xaa₃ is Glu or Asp; Xaa₄ is Ala, Gly, Ile, Leu, Met, or Val; Xaa₆ is Ala, Gly, Ile, Leu, Met, or Val; Xaa₇ is Glu or Asp; Xaa₈ is Asp or Glu; Xaa₉ is Arg, His, or Lys; Xaa₁₀ is Ser, Asn, Gln, or Thr; Xaa₁ is Leu, Ala, Gly, Ile, Leu, Met or Val; Xaa₁₂ is Gly, is any amino acid residue of the D-configuration, or is not present; Xaa₁₃ is Arg, His, or Lys or is not present; Xaa₁₄ is Arg, His, or Lys or is not present.

An exemplary peptide conforming to Formula II can have the sequence Pro-Pro-Glu-Ala-Pro-Ala-Glu-Asp-Arg-Ser-Leu-Gly-Arg-Arg (SEQ ID NO:2).

In another embodiment, the fragments of a GDNF precursor protein or the biologically active variants can have, or can include, a sequence of amino acid residues conforming to the amino acid sequence of Formula III:

(SEQ ID NO:39)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃-Xaa₁₄-Xaa₁₅-Xaa₁₆-Xaa₁₇-
Xaa₁₈-Xaa₁₉-Xaa₂₀-Xaa₂₁-Xaa₂₂ (III).

Within this Formula, Xaa₁ and Xaa₂ are, independently, Arg, Lys, or H is or are absent; Xaa₃ is Glu or Asp; Xaa₄ is Arg, Lys, or His; Xaa₅ is Asn, Gln, Ser, or Thr; Xaa₆ is Arg, Lys, or His; Xaa₇ is Gln, Asn, Ser, or Thr; Xaa₈, Xaa₉, Xaa₁₀, and Xaa₁₁ are, independently, Ala, Gly, Ile, Leu, Met, or Val; Xaa₁₂ is Asn, Gln, Ser, or Thr; Xaa₁₃ is Pro or Ser; Xaa₁₄ is Glu or Asp; Xaa₁₅ is Asn, Gln, Ser, or Thr; Xaa₁₆ is Ser, Asn, Gln, or Thr; Xaa₁₇ is Lys, Arg, or His; Xaa₁₈ is Gly, Ala, Ile, Leu, Met, or Val; Xaa₁₉ is Lys, Arg, or His; Xaa₂₀ is Gly, is any amino acid residue of the D-configuration, or is not present; and Xaa₂₁ and Xaa₂₂ are, independently, Arg, Lys, His, or are not present.

An exemplary peptide conforming to Formula III can have the sequence Arg-Arg-Glu-Arg-Asn-Arg-Gln-Ala-Ala-Ala-Ala-Asn-Pro-Glu-Asn-Ser-Arg-Gly-Lys-Gly-Arg-Arg (SEQ ID NO:3).

The amino acid residues, in any embodiment, can be of the D- or L-form or a combination thereof. In some embodiments, for example, SEQ ID NO's 4, 5 and 6, the polypeptides may exhibit reduced antigenic propensity.

The polypeptides of the invention can also include structural modifications, which may be introduced to, for example, modulate therapeutic efficacy of the polypeptides or to assist clinicians in monitoring a course of treatment with the polypeptides. Structural modifications can be made during or after polypeptide translation or chemical synthesis. For example, the fragment of a GDNF precursor protein or the biologically active variant can be amidated. Alternatively, or in addition, the fragment of a GDNF precursor protein can include a detectable marker. Both the form and position of the detectable marker can vary, as long as the polypeptide retains a sufficient amount of the biological activity of the unmarked polypeptide to remain useful. Suitable markers include, for example, photo-affinity ligands, radio-isotopes, and fluorescent or chemiluminescent compounds.

The invention also features isolated nucleic acids that have, or that include, a sequence encoding any of the above described polypeptides. Recombinant vectors are also provided. A recombinant vector can include a nucleotide sequence encoding any of the described polypeptide fragments. The nucleotide sequence can be operably linked to a regulatory region suitable for use in either a prokaryotic or a eukaryotic system, many of which are known in the art. In specific embodiments, the regulatory region can be, for example, a promoter. Useful promoters include cell type-specific promoters, tissue-specific promoters, constitutively active promoters and broadly expressing promoters. Host cells including such nucleic acid constructs are also provided.

The polypeptides and nucleic acids can be formulated as pharmaceutical compositions. For example, the compositions can include polypeptides having only one of the specific amino acid sequences listed in either Formulas I, II, or III, or they can include mixtures of the variants described for Formulas I, II, or III. For example, the compositions can include mixtures of polypeptides conforming to Formula I; mixtures of polypeptides conforming to Formula II; mixtures of polypeptides conforming to Formula III; mixtures of polypeptides conforming to Formulas I and II; mixtures of polypeptides conforming to Formulas II and III; mixtures of polypeptides conforming to Formulas I and III; or mixtures of polypeptides conforming to Formulas I, II, and III. Where nucleic acids are formulated as pharmaceutical compositions, the nucleic acids can similarly encode polypeptides in the configurations just described. The polypeptides of the invention can be formulated in any pharmaceutically acceptable medium. Carriers and stabilizing agents may be added to facilitate drug delivery and to insure shelf-life. For example, encapsulation of the polypeptides in a suitable delivery vehicle (e.g., polymeric microparticles or implantable devices) may increase the efficiency of delivery.

The methods of the invention include methods for treating a subject (e.g., a human patient) with a neurological disorder. These methods can include the steps of a) identifying a subject who is experiencing or is likely to experience a neurological disorder; and b) providing to the subject a composition including fragments of GDNF precursor proteins or biologically active variants of GDNF precursor proteins. The compositions can be administered to a subject in a variety of ways. For example, the compositions can be administered transdermally or injected (infused) intravenously, subcutaneously, intracranially, intramuscularly, intraperitoneally, or intrapulmonarily. Oral formulations are also within the scope of the present invention. The dosage required will depend upon various factors typically considered by one of ordinary skill in the art. These factors include the route of administration, the nature of the formulation, the nature of the patient's illness, the subject's size, weight, surface area, age, gender, other drugs being administered to the patient, and the judgement of the attending physician. Suitable dosages are in the range of 0.01-100.0 μg/kg. The compositions can be administered along with or in addition to standard treatments for particular neurological disorders, e.g., drug therapy, immunotherapy, or surgery.

Neurological disorders amenable to the diagnostic, therapeutic, and/or prognostic methods of the invention can include disorders of both motor and sensory neurons and may be the result of disease, injury, genetic factors or unknown causes. While we believe we understand certain events that occur in the course of treatment, the compositions of the present invention are not limited to those that work by affecting any particular cellular mechanism. Motor system disorders include for example, but are not limited to, Parkinson's Disease, ataxia, dyskinesia, Multiple System Atrophy, Shy-Drager Syndrome, Progressive Supranuclear Palsy and Essential Tremor. Sensory neuron disorders include hearing disorders (e.g., disorders related to defects of, or trauma to, inner ear sensory cells). In another embodiment, the compositions and methods provided herein can be used to treat neurologic syndromes, (e.g., attention deficit disorders such as ADHD and addictions, such as addictions to a drug, whether illicit or prescribed (including alcohol and nicotine)).

The compositions described herein can also be assembled in kits, together with instructions for use. For example, the kits can include measured amounts of a pharmaceutical composition that includes one or more of the polypeptides described herein, or a biologically active variant thereof, and packaging materials.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 depicts the amino acid sequence of human glial-cell derived neurotropic factor (GDNF) (SEQ ID NO:8).

FIG. 2 depicts amino acid sequence alignments of fragments of human GDNF precursor proteins with homologous segments from other species.

FIG. 3 is a graph depicting the effect of GDNF-derived peptide FPLPA (SEQ ID NO:4) on calcium mobilization in C6 cells.

FIG. 4 is a graph depicting the effect of GDNF-derived peptide PPEAPAEDRSL (SEQ ID NO:5) on calcium mobilization in C6 cells.

FIG. 5 is a graph depicting the effect of combinations of GDNF-derived peptides (peptide #1 is SEQ ID NO:5; peptide #2 is SEQ ID NO:6; and peptide #3 is SEQ ID NO:4) on calcium mobilization in C6 cells in calcium-free buffer.

FIG. 6 is a graph depicting the effect of combinations of GDNF-derived peptides (peptide #1 is SEQ ID NO:5; peptide #2 is SEQ ID NO:6; and peptide #3 is SEQ ID NO:4) on calcium mobilization in C6 cells in calcium-containing buffer.

### DETAILED DESCRIPTION

Glial-derived neurotrophic factor (GDNF) is a protein that promotes the survival of several types of neuronal cells, and it has also been referred to in the art as glial cell line-derived neurotrophic factor, glial cell line-derived neurotrophic factor precursor, and astrocyte-derived trophic factor. GDNF is initially expressed as a precursor protein of about 211 amino acids that is processed by cellular enzymes to a mature species of about 133 amino acids. The native form of biologically active GDNF is secreted as a glycosylated disulfide-linked homodimer composed of two molecules of the mature polypeptide. GDNF is particularly effective in promoting the survival of dopaminergic and motor neurons, which are the populations of cells that degenerate in Parkinson's disease and amyotrophic lateral sclerosis (ALS; also known as Lou Gehrig's Disease), respectively. Disclosed herein are materials and methods related to the production and use of fragments of a GDNF precursor protein for the treatment and management of neurological disorders, including those that involve GDNF-responsive neurons (e.g., dopaminergic or motor neurons).

Compositions

Polypeptides

The terms “polypeptide” and “peptide” are used herein to refer to a compound of two or more subunit amino acids, amino acid analogs, or other peptidomimetics, regardless of post-translational modification (e.g., amidation, phosphorylation or glycosylation). The subunits can be linked by peptide bonds or other bonds such as, for example, ester or ether bonds. The term “amino acid” refers to natural and/or unnatural or synthetic amino acids, which may, as noted above, be D- or L-form optical isomers.

The cDNA and amino acid sequences encoding representative human GDNF nucleotide and polypeptide sequences (Genbank number NP_{—}000505.1, public GI:4503975) are shown in Examples 8 (SEQ ID NO:7) and 9 (SEQ ID NO:8), respectively. Other representative forms of GDNF can have an amino acid sequence that has 1, 2, 3, 4, 5, 10 or more amino acid changes compared to the amino acid sequence of Genbank number NP_{—}000505.1, public GI:4503975. Other amino acid sequences that have been identified for GDNF include for example, without limitation, NP_{—}954701.1, public GI:40549411; NP_{—}954704.1, public GI:40549413; CAG46721.1, public GI:49456801; AA128109.1, public GI:118763995; and P39905, public GI:729567; NM_{—}000514.2, public GI:40549401.

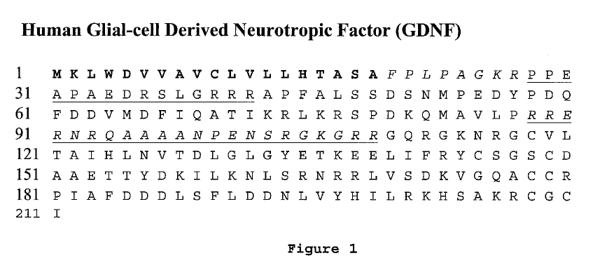

The polypeptides described herein include fragments of a GDNF precursor protein and biologically active variants thereof. A fragment of a GDNF precursor protein can include a fragment of a native GDNF precursor protein as shown in FIG. 1 (e.g., SEQ ID NO:8) and/or a processed and mature GDNF. While the sequences of the fragments of a GDNF precursor protein can vary (e.g., in length), useful fragments of a GDNF precursor protein can include not more than 92 amino acid residues and can exclude some or all of amino acid residues 118-211 of SEQ ID NO:8. Thus, a fragment of a GDNF precursor protein can be 2-5 amino acids long (e.g., 4 amino acids long); 5-10 amino acids long (e.g., 8 amino acids long); 10-20 amino acids long (e.g., 18 amino acids long); 20-40 amino acids long (e.g., 22, 23, 24, 25, 30, or 35 amino acids long; 40-60 amino acids long (e.g., 50 amino acids long); 60-80 amino acids long (e.g., 70 amino acids long); or 80-92 amino acids long (e.g. 85 amino acids long).

A fragment of a GDNF precursor protein can have or can include an amino acid sequence naturally expressed (e.g., in a full length GDNF protein) in a human cell. Alternatively, a fragment of a GDNF precursor protein can have or can include an amino acid sequence of a fragment of a homolog or ortholog of the polypeptide having the amino acid sequence set forth in SEQ ID NO: 8. As noted, the present invention includes biologically active variants of fragments of a GDNF precursor protein, and these variants can have or can include, for example, an amino acid sequence that differs from a wildtype fragment of a GDNF protein (e.g., a precursor protein or mature GDNF protein) by virtue of containing one or more mutations (e.g., an addition, deletion, or substitution mutation or a combination of such mutations). One or more of the substitution mutations can be a substitution (e.g., a conservative amino acid substitution), with the proviso that at least or about 50% of the amino acid residues of the variant are identical to residues in the corresponding wildtype fragment of a GDNF protein. For example, a biologically active variant of a GDNF polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a GDNF protein (e.g., to the amino acid sequence set forth in SEQ ID NO:8 or to a homolog or ortholog thereof).

Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine.

More specifically, GDNF polypeptides or biologically active fragments thereof can have, or can include, a sequence of amino acid residues conforming to one or more of Formulas I, II and III. Locations of the GDNF peptides of Formula I, II and III within a GDNF precursor protein (SEQ ID NO:8) are indicated by italicized text, underlined text, and italicized and underlined text, respectively, in FIG. 1.

Thus, for example, a fragment of a GDNF precursor protein or a biologically active fragment thereof can have, or can include, a sequence of amino acid residues conforming to the amino acid sequence of Formula I:

(SEQ ID NO:9)

Xaa₁-Pro-Xaa₃-Pro-Xaa₅-Xaa₆-Xaa₇-Xaa₈ (I) wherein

Xaa₁ is Phe, Trp, or Tyr;

Xaa₃ is Leu, Ala, Ile, or Val;

Xaa₅ is Ala, Leu, Ile, or Val;

Xaa₆ is Gly, is any amino acid residue of the D configuration, or is not present

Xaa₇ is Lys, Arg, or His; or is not present

Xaa₈ is Arg, Lys, or His; or is not present,

wherein Pro represents the amino acid proline and each Xaa (e.g., Xaa₁) represents an amino acid residue at the positions indicated by the subscript.

In specific embodiments, the fragment of a GDNF precursor protein of Formula I can conform to the following specifications. The peptide can have a sequence represented by: (1) Phe-Pro-Xaa₃-Pro-Xaa₅-Xaa₆-Xaa₇-Xaa₈ (SEQ ID NO:10), where Xaa₃ and Xaa₅-Xaa₆-Xaa₇-Xaa₈ are, independently, as described above (e.g., Phe-Pro-Leu-Pro-Ala-Gly-Lys-Arg (SEQ ID NO:1)); (2) Xaa₁-Pro-Leu-Pro-Xaa₅-Xaa₆-Xaa₇-Xaa₈ (SEQ ID NO:1), where Xaa₁, and Xaa₅-Xaa₆-Xaa₇-Xaa₈ are, independently, as described above; (3) Phe-Pro-Leu-Pro-Xaa₅-Xaa₆-Xaa₇-Xaa₈ (SEQ ID NO:12), where Xaa₅-Xaa₆-Xaa₇-Xaa₈ are, independently, as described above; (4) Xaa₁-Pro-Xaa₃-Pro-Ala-Xaa₆-Xaa₇-Xaa₈ (SEQ ID NO:13), where Xaa₁, Xaa₃ and Xaa₆-Xaa₇-Xaa₈ are, independently, as described above; (5) Phe-Pro-Xaa₃-Pro-Ala-Xaa₆-Xaa₇-Xaa₈ (SEQ ID NO:14), where Xaa₃ and Xaa₆-Xaa₇-Xaa₈ are, independently, as described above; (6) Phe-Pro-Leu-Pro-Ala-Xaa₆-Xaa₇-Xaa₈ (SEQ ID NO:15), where Xaa₆, Xaa₇, and Xaa₈ are, independently, as described above; (7) Xaa₁-Pro-Xaa₃-Pro-Xaa₅-Gly-Xaa₇-Xaa₈ (SEQ ID NO:16), where Xaa₁, Xaa₃, Xaa₅, and Xaa₇-Xaa₈ are, independently, as described above; (8) Phe-Pro-Xaa₃-Pro-Xaa₅-Gly-Xaa₇-Xaa₈ (SEQ ID NO:17), where Xaa₃, Xaa₅, and Xaa₇-Xaa₈ are, independently, as described above; (9) Phe-Pro-Leu-Pro-Xaa₅-Gly-Xaa₇-Xaa₈ (SEQ ID NO:18), where Xaa₅, and Xaa₇-Xaa₈ are, independently, as described above; (10) Phe-Pro-Leu-Pro-Ala-Gly-Xaa₇-Xaa₈ (SEQ ID NO:19), where Xaa₇ and Xaa₈ are, independently, as described above; (11) Xaa₁-Pro-Xaa₃-Pro-Xaa₅-Xaa₆-Lys-Xaa₈ (SEQ ID NO:20), where Xaa₁, Xaa₃, Xaa₅, Xaa₆ and Xaa₈ are, independently, as described above; (12) Phe-Pro-Xaa₃-Pro-Xaa₅-Xaa₆-Lys-Xaa₈ (SEQ ID NO:21), where Xaa₃, Xaa₅, Xaa₆ and Xaa₈ are, independently, as described above; (13) Phe-Pro-Leu-Pro-Xaa₅-Xaa₆-Lys-Xaa₈ (SEQ ID NO:22), where Xaa₅, Xaa₆ and Xaa₈ are, independently, as described above; (14) Phe-Pro-Leu-Pro-Ala-Xaa₆-Lys-Xaa₈ (SEQ ID NO:23), where Xaa₆ and Xaa₈ are, independently, as described above; (15) Phe-Pro-Leu-Pro-Ala-Gly-Lys-Xaa₈ (SEQ ID NO:24), where Xaa₈ is as described above; (16) Xaa₁-Pro-Xaa₃-Pro-Xaa₅-Xaa₆-Xaa₇-Arg (SEQ ID NO:25) where Xaa₁, Xaa₃, Xaa₅, Xaa₆ and Xaa₇ are, independently, as described above; (17) Phe-Pro-Xaa₃-Pro-Xaa₅-Xaa₆-Xaa₇-Arg (SEQ ID NO:26) where Xaa₃, Xaa₅, Xaa₆ and Xaa₇ are, independently, as described above; (18) Phe-Pro-Leu-Pro-Xaa₅-Xaa₆-Xaa₇-Arg (SEQ ID NO:27) where Xaa₅, Xaa₆ and Xaa₇ are, independently, as described above; (19) Phe-Pro-Leu-Pro-Ala-Xaa₆-Xaa₇-Arg (SEQ ID NO:28), where Xaa₆ and Xaa₇ are, independently, as described above; (20) Phe-Pro-Leu-Pro-Ala-Gly-Xaa₇-Arg (SEQ ID NO:29), where Xaa₇ is as described above; and so forth. In a more specific embodiment, the fragment of a GDNF precursor protein can be a fragment or portion of a GDNF precursor protein conforming to Formula I, where Xaa₁ is Phe, Xaa₃ is Leu, Xaa₅ is Ala, Xaa₆ is Gly, Xaa₇ is Lys and Xaa₈ is Arg (i.e., Phe-Pro-Leu-Pro-Ala-Gly-Lys-Arg (SEQ ID NO:1)). At least one (e.g., one, two, or three) of the amino acid residues represented by Formula I can be absent. For example, Xaa₁, Xaa₇, and/or Xaa₈ can be absent.

In another embodiment, the fragments of a GDNF precursor protein or the biologically active variants thereof can have, or can include, a sequence of amino acid residues conforming to the amino acid sequence of Formula II:

(SEQ ID NO:30)

Pro-Pro-Xaa₃-Xaa₄-Pro-Xaa₆-Xaa₇-Xaa₈-Xaa₉-Xaa₁₀-
Xaa₁₁-Xaa₁₂-Xaa₁₃-Xaa₁₄ (II) wherein

Xaa₃ is Glu or Asp;

Xaa₄ is Ala, Gly, Ile, Leu, Met, or Val;

Xaa₆ is Ala, Gly, Ile, Leu, Met, or Val;

Xaa₇ is Glu or Asp;

Xaa₈ is Asp or Glu;

Xaa₉ is Arg, His, or Lys;

Xaa₁₀ is Ser, Asn, Gln, or Thr;

Xaa₁₁ is Leu, Ala, Gly, Ile, Leu, Met or Val;

Xaa₁₂ is Gly, is any amino acid residue of the D-configuration, or is not present;

Xaa₁₃ is Arg, His, or Lys or is not present;

Xaa₁₄ is Arg, His, or Lys or is not present;

wherein Xaa₁₋₁₄ represents an amino acid and the subscripts 1-14 represent the positions of each amino acid in the peptide sequence.

In specific embodiments, the fragments of a GDNF precursor protein of Formula II can conform to the following specifications. The peptide can have a sequence represented by, for example: (1) Pro-Pro-Xaa₃-Xaa₄-Pro-Xaa₆-Xaa₇-Xaa₈-Xaa₉-Xaa₁₀-Xaa₁₁-Xaa₁₂-Xaa₁₃-Xaa₁₄ (SEQ ID NO:30), where Xaa₃-Xaa₄ and Xaa₆-Xaa₇-Xaa₈-Xaa₉-Xaa₁₀-Xaa₁₁-Xaa₁₂-Xaa₁₃-Xaa₁₄ are, independently, as described above (e.g., Pro-Pro-Glu-Ala-Pro-Ala-Glu-Asp-Arg-Ser-Leu-Gly-Arg-Arg, SEQ ID NO:2); (2) Pro-Pro-Glu-Xaa₄-Pro-Xaa₆-Xaa₇-Xaa₈-Xaa₉-Xaa₁₀-Xaa₁-Xaa₁₂-Xaa₁₃-Xaa₁₄ (SEQ ID NO:31), where Xaa₄ and Xaa₆-Xaa₇-Xaa₈-Xaa₉-Xaa₁₀-Xaa₁₁-Xaa₁₂-Xaa₁₃-Xaa₁₄ are, independently, as described above; (3) Pro-Pro-Asp-Xaa₄-Pro-Xaa₆-Xaa₇-Xaa₈-Xaa₉-Xaa₁₀-Xaa₁₁-Xaa₁₂-Xaa₁₃-Xaa₁₄ (SEQ ID NO:32), where Xaa₄ and Xaa₆-Xaa₇-Xaa₈-Xaa₉-Xaa₁₀-Xaa₁₁-Xaa₁₂-Xaa₁₃-Xaa₁₄ are, independently, as described above; (4) Pro-Pro-Glu-Xaa₄-Pro-Xaa₆-Glu-Xaa₈-Xaa₉-Xaa₁₀-Xaa₁₁-Xaa₁₂-Xaa₁₃-Xaa₁₄ (SEQ ID NO:33), where Xaa₄ and Xaa₆-Xaa₈-Xaa₉-Xaa₁₀-Xaa₁₁-Xaa₁₂-Xaa₁₃-Xaa₁₄ are, independently, as described above; (5) Pro-Pro-Glu-Xaa₄-Pro-Xaa₆-Asp-Xaa₈-Xaa₉-Xaa₁₀-Xaa₁₁-Xaa₁₂-Xaa₁₃-Xaa₁₄ (SEQ ID NO:34), where Xaa₄ and Xaa₆-Xaa₈-Xaa₉-Xaa₁₀-Xaa₁₁-Xaa₁₂-Xaa₁₃-Xaa₁₄ are, independently, as described above; (6) Pro-Pro-Glu-Xaa₄-Pro-Xaa₆-Glu-Asp-Xaa₉-Xaa₁₀-Xaa₁₁-Xaa₁₂-Xaa₁₃-Xaa₁₄ (SEQ ID NO:35), where Xaa₄ and Xaa₆-Xaa₉-Xaa₁₀-Xaa₁₁-Xaa₁₂-Xaa₁₃-Xaa₁₄ are, independently, as described above; (7) Pro-Pro-Glu-Xaa₄-Pro-Xaa₆-Glu-Glu-Xaa₉-Xaa₁₀-Xaa₁₁-Xaa₁₂-Xaa₁₃-Xaa₁₄ (SEQ ID NO:36), where Xaa₄ and Xaa₆-Xaa₉-Xaa₁₀-Xaa₁₁-Xaa₁₂-Xaa₁₃-Xaa₁₄ are, independently, as described above; (8) Pro-Pro-Glu-Xaa₄-Pro-Xaa₆-Asp-Asp-Xaa₉-Xaa₁₀-Xaa₁₁-Xaa₁₂-Xaa₁₃-Xaa₁₄ (SEQ ID NO:37), where Xaa₄ and Xaa₆-Xaa₉-Xaa₁₀-Xaa₁₁-Xaa₁₂-Xaa₁₃-Xaa₁₄ are, independently, as described above; (9) Pro-Pro-Glu-Xaa₄-Pro-Xaa₆-Asp-Glu-Xaa₉-Xaa₁₀-Xaa₁₁-Xaa₁₂-Xaa₁₃-Xaa₁₄ (SEQ ID NO:38), where Xaa₄ and Xaa₆-Xaa₉-Xaa₁₀-Xaa₁₁-Xaa₁₂-Xaa₁₃-Xaa₁₄ are, independently, as described above; and so forth. At least one (e.g., one, two, or three) of the amino acid residues represented by Formula II can be absent. For example, Xaa₁₂, Xaa₁₃, and/or Xaa₁₄ can be absent. Other variant sequences, not specifically listed here, will be readily apparent, given the present formulas, the present provisions, and examples such as these, to one of ordinary skill in the art.

In another embodiment, the fragments of GDNF precursor proteins or the biologically active variants can have, or can include, a sequence of amino acid residues conforming to the amino acid sequence of Formula III:

(SEQ ID NO:39)

Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃-Xaa₁₄-Xaa₁₅-Xaa₁₆-Xaa₁₇-
Xaa₁₈-Xaa₁₉-Xaa₂₀-Xaa₂₁-Xaa₂₂ (III),

wherein

Xaa₁ and Xaa₂ are, independently, Arg, Lys, or His;

Xaa₃ is Glu or Asp;

Xaa₄ is Arg, Lys, or His;

Xaa₅ is Asn, Gln, Ser, or Thr;

Xaa₆ is Arg, Lys, or His;

Xaa₇ is Gln, Asn, Ser, or Thr;

Xaa₈ Xaa₉, Xaa₁₀, and Xaa₁₁ are, independently, Ala, Gly, Ile, Leu, Met, or Val;

Xaa₁₂ is Asn, Gln, Ser, or Thr;

Xaa₁₃ is Pro or Ser;

Xaa₁₄ is Glu or Asp;

Xaa₁₅ is Asn, Gln, Ser, or Thr;

Xaa₁₆ is Ser, Asn, Gln, or Thr;

Xaa₁₇ is Lys, Arg, or His;

Xaa₁₈ is Gly, Ala, Ile, Leu, Met, or Val;

Xaa₁₉ is Lys, Arg, or His;

Xaa₂₀ is Gly, is any amino acid residue of the D-configuration, or is not present;

Xaa₂₁ and Xaa₂₂ are, independently, Arg, Lys, His, or are not present;

In the formulas, amino acid residues are represented by the standard three-letter code. Where a variety of residues may be used, the amino acid is represented by Xaa, and the subscript represents the position of each amino acid in the peptide sequence.

In specific embodiments, the fragments of a GDNF precursor protein of Formula III can conform to the following specifications. The peptide can have a sequence represented by, for example: (1) Arg-Arg-Glu-Arg-Asn-Arg-Gln-Ala-Ala-Ala-Ala-Asn-Pro-Glu-Asn-Ser-Arg-Gly-Lys-Gly-Arg-Arg, (SEQ ID NO:3) (2) Arg-Arg-Glu-Arg-Asn-Arg-Gln-Ala-Ala-Ala-Ala-Asn-Pro-Glu-Asn-Ser-Lys-Gly-Lys-Gly-Arg-Arg (SEQ ID NO:40); (3) Arg-Arg-Glu-Arg-Asn-Arg-Gln-Ala-Ala-Ala-Ala-Ser-Pro-Glu-Asn-Ser-Arg-Gly-Lys-Gly-Arg-Arg (SEQ ID NO:41); (4) Lys-Arg-Glu-Arg-Asn-Arg-Gln-Ala-Ala-Ala-Ala-Asn-Pro-Glu-Asn-Ser-Arg-Gly-Lys-Gly-Arg-Arg (SEQ ID NO:42); (5) Arg-Lys-Glu-Arg-Asn-Arg-Gln-Ala-Ala-Ala-Ala-Asn-Pro-Glu-Asn-Ser-Arg-Gly-Lys-Gly-Arg-Arg (SEQ ID NO:43); (6) Arg-His-Glu-Arg-Asn-Arg-Gln-Ala-Ala-Ala-Ala-Asn-Pro-Glu-Asn-Ser-Arg-Gly-Lys-Gly-Arg-Arg (SEQ ID NO:44); (7) Arg-His-Glu-Arg-Asn-Arg-Gln-Ala-Ala-Ala-Ala-Asn-Pro-Glu-Asn-Ser-Arg-Gly-Lys-Gly-Arg-Arg (SEQ ID NO:44); (8) His-His-Glu-Arg-Asn-Arg-Gln-Ala-Ala-Ala-Ala-Asn-Pro-Glu-Asn-Ser-Arg-Gly-Lys-Gly-Arg-Arg (SEQ ID NO:45); and so forth. At least one (e.g., one, two, or three) of the amino acid residues represented by Formula III can be absent. For example, Xaa₁, Xaa₂, Xaa₂₀, Xaa₂₁, and/or Xaa₂₂ can be absent. Other variant sequences, not specifically listed here, will be readily apparent, given the present formulas, the present provisions, and examples such as these, to one of ordinary skill in the art.

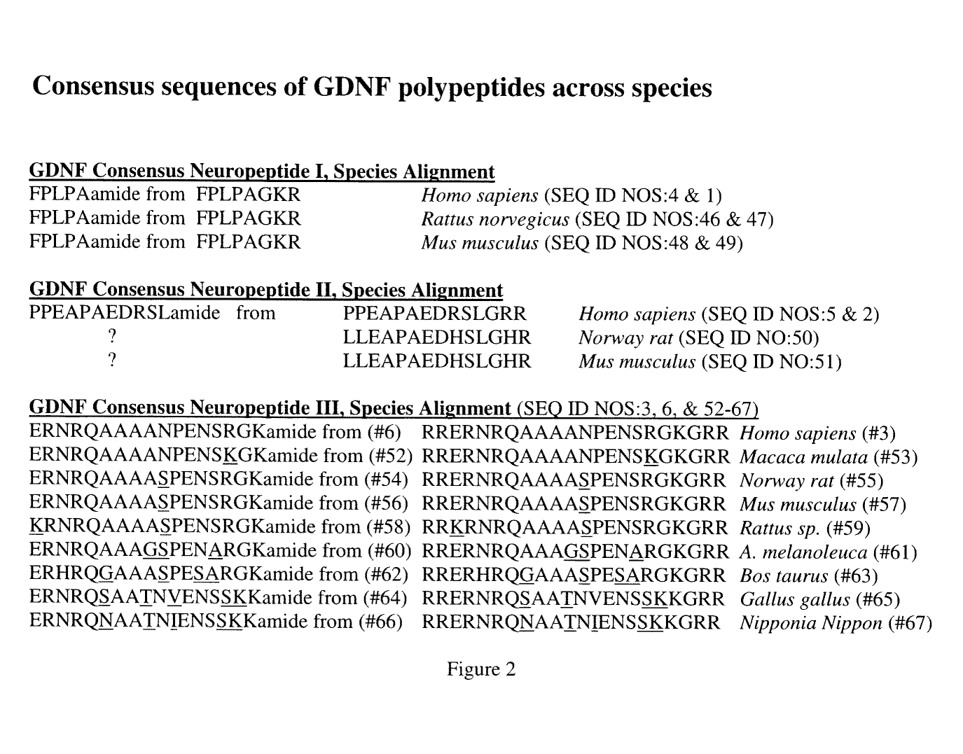

Amino acid sequences of homologs and/or orthologs of fragments of a GDNF precursor protein conforming to Formulas I, II and III are provided in FIG. 2, along with a consensus sequence. A consensus amino acid sequence for such homologs and/or orthologs was determined by aligning amino acid sequences (e.g., amino acid sequences related to SEQ ID NO: 8), from a variety of species and determining the most common amino acid or type of amino acid at each position. For example, the alignment in FIG. 2 provides the amino acid sequences of the fragment of a human GDNF precursor protein of formula I with homologous sequences from Rattus norvegicus and Mus musculus; the amino acid sequences of the fragment of a human GDNF precursor protein of formula II with homologous sequences from Rattus norvegicus and Mus musculus; and the amino acid sequences of the fragment of a human GDNF precursor protein of formula III with homologous sequences from Macaca mulata, Rattus norvegicus, Mus musculus, Rattus spp., Ailuropoda melanoleuca, Bos taurus, Gallus gallus and Nipponia nippon.

The amino acid residues in biologically active variants of the fragments of a GDNF precursor protein can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g. pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine(2R,3S)-2-amino-3-methylpentanoic acid and L-cyclopentyl glycine (S)-2-amino-2-cyclopentyl acetic acid. For other examples, one can consult textbooks or the worldwide web (a site is currently maintained by the California Institute of Technology and displays structures of non-natural amino acids that have been successfully incorporated into functional proteins). Non-natural amino acid residues and amino acid derivatives listed in U.S. Application No. 20040204561 (see ¶0042, for example) can also be used.

As noted, the invention encompasses biologically active variants of fragments of a GDNF precursor protein. A biologically active variant will have one or more of the biological activities of a cognate fragment of a GDNF precursor protein. These activities include those described herein as well as the biological activities of a GDNF precursor protein, a mature GNDF protein, or a secreted homodimer (e.g., a secreted human homodimer). The biological activities can include, without limitation, GDNF receptor activation, stimulation of neuronal growth, prolongation of neuronal survival, inhibition of neuronal degeneration, modulation (e.g., induction) of neuronal cell differentiation, and protection of neurons against toxins (e.g., excitotoxic agents). A variety of assays and methods can be employed to assess the biological activity of any given polypeptide. Biologically active variants of fragments of a GDNF precursor protein can be identified, for example, by comparing the relative activities of the variant polypeptide with that of a native form of a GDNF polypeptide or a fragment thereof. One could also test, if desired, an unrelated control polypeptide (e.g. one could include in any given assay a peptide that has the same amino acid content randomly arranged). A biologically active variant of a fragment of a GDNF precursor protein will retain sufficient biological activity to be useful in the methods described herein (e.g., in a method of treating a patient). Some biologically active variants may even have greater biological activity than the cognate, naturally occurring fragment. More specifically, a biologically active variant of GDNF can have at least or about 30%, 40%, 50%, 60%, 70%, 80%, 90%, 95%, 98%, or more of the biological activity of the native form of the GDNF polypeptide or fragment thereof. The methods that can be used to assess activity include in silico analyses, in vitro assays, cell-based assays and whole animal, in vivo, model systems. These assays can be configured to test the effect of any given fragment of a GDNF precursor protein, or a variant thereof, on processes such as cAMP modulation, wound healing, cell proliferation, cell survival (e.g., a cell protection assay), and cell migration. Fragments of a GDNF precursor protein and variants thereof can also be compared with GDNF proteins for utility in receptor distribution mapping.

Useful cell-based assays include those that examine activation of the G-protein coupled receptor complex for GDNF. The complex includes the receptor tyrosine complex, RET, which functions as a signaling module, and the GDNF family receptor α (GFRα), a cell-surface-bound co-receptor. G-protein coupled receptors convert the peptide-receptor recognition signal into a small number of different “second messengers” (e.g., calcium fluxes across membranes, cyclic nucleotides, and phosphoinositides). Thus, modulation in the level or activity of one or more second messengers in GDNF-treated cells relative to the corresponding levels in control (e.g., untreated) cells can be indicative of activation of the G-protein coupled receptor complex for GDNF, and assessing GDNF, fragments of a GDNF precursor protein, and variants thereof indicates the relative levels of biological activity each agent possesses.

Assessing second messenger activity is routine in the art, and kits and reagents for performing such assays are readily available from commercial sources. For example, alterations in calcium levels can be assayed with calcium-sensitive dyes such as Calcium Crimson-AM (Invitrogen, Carlsbad, Calif.), FLIPR (Molecular Devices, Sunnyvale, Calif.), Fluo4 and Fura Red (Caliper Life Sciences, Hopkinton, Mass.), which can be monitored either by microscopy or fluorometry. Modulation of cyclic nucleotides can be assayed by chemiluminescence, immunoassays, or fluorescence polarization techniques. Changes in phosphoinisitide levels can be evaluated by fluorescence polarization, immunoassays and other downstream markers such as D-myo-inositol 1-phosphate.

Peptide activity can also be monitored in cell-based assays that measure the impact of GDNF, fragments of a GDNF precursor protein, and/or variants thereof on specific cell functions and/or on cell survival generally. Methods of measuring various cell functions and survival are well-known in the art. In the context of the present invention, suitable assays include, without limitation, in vitro wound healing assays, cell proliferation assays, assays that measure cell protection from exogenous neurotoxic agents (e.g., 6-hydroxydopamine (6-OHDA), 1-methyl-4-phenyl-1,2,3,6-tetrahydropyridine (MPTP), and kainite), and cell migration assays.

Any cell type that is responsive to GDNF, or any tissue containing GDNF-responsive cells, can be used to assess biological activity, including cell lines and explants containing GDNF-responsive neuronal cells. Particularly useful cell lines include the neuronal cell lines C6, N1E and PC 12. Cell lines can be obtained from standard commercial sources and from depositories such as The American Type Culture Collection. Alternatively, or in addition, cells or cell lines that have been genetically engineered to express GDNF receptors can be used. Neuronal explant systems for evaluating GDNF can include microcultures as described, for example, by Hsu et al. (Methods in Cell Science 7:143-145, 2005). Chemoattractant functions can be tested as described by Paratcha (Mol. Cell. Neurosci. 31:505-514, 2006) using neuronal precursor cells, and ciliary ganglion cell explants can be used to examine neurite outgrowth and survival of that cell type. GDNF availability determines enteric neuron number by controlling ENS precursor proliferation. Effects on cell survival can be tested using, for example, enteric neurons (see Giamino et al., Development 130:2187-2198, 2003). As GDNF promotes the survival of sympathetic, parasympathetic and spinal motor neurons during development, protects midbrain dopaminergic neurons from apoptosis, and influences renal and testicular development, these cell types can also be used to determine the activity of a fragment of a GDNF precursor protein or a variant thereof. These cell types and assays serve to illustrate means by which the present peptides can be tested. One of ordinary skill in the art will recognize additional and appropriate assays. The activity of the fragments of a GDNF precursor protein can also be evaluated in vivo. Suitable animal model systems for Parkinson's disease have been developed in both Drosophila melanogaster and mice and include, for example, without limitation, systems described in Feany M. B and Bender W. W. (2000). Nature 404 (6776), 394-8; Auluck P K, Chan H Y, Trojanowski J Q, Lee V M, and Bonini N M. Science 2002, February 1; 295(5556): 809-100); J Neurosci. 22(20):8797, 2002; Neurobiol Aging, 2005 January; 26(1):25-35; Proc Natl Acad Sci USA, 2005 Feb. 8; 102(6):2174-9; Mol Cell Neurosci, 24:419, 2003).

Fragments of a GDNF precursor protein and biologically active variants thereof can be chemically synthesized, purified from natural sources (insofar as they exist in those sources or can be obtained from naturally occurring proteins (by, for example, digestion)), or purified from cells in which the fragment of a GDNF precursor protein or a biologically active variant thereof is recombinantly produced. The methods required for peptide synthesis, expression and purification are well known in the art. For example, peptides can be chemically synthesized using standard f-moc chemistry and purified using high pressure liquid chromatography (HPLC). Fragments of a GDNF precursor protein and biologically active variants thereof can be purified by any method known in the art, including without limitation, fractionation, centrifugation, and chromatography (e.g., gel filtration, ion exchange chromatography, reverse-phase HPLC and immunoaffinity purification).

Purified peptides may be, but are not necessarily, highly purified. A fragment of a GDNF precursor protein or a biologically active variant thereof will be substantially pure when it has been separated from the components (e.g., cellular components) with which it was previously associated such that a composition in which it is contained is at least or about 60% (e.g., at least or about 70%, 80%, 90%, 95%, or 99%), by weight, the fragment of a GDNF precursor protein or the biologically active variant thereof. In general, a substantially pure polypeptide will yield a single major band on a non-reducing polyacrylamide gel.

To produce a recombinant fragment of a GDNF precursor protein or a biologically active variant thereof, a nucleic acid encoding the peptide can be ligated into an expression vector and used to transform a prokaryotic (e.g., bacteria) or transfect a eukaryotic (e.g., insect, yeast, or mammal) host cell. In general, nucleic acid constructs can include a regulatory sequence operably linked to a nucleic acid encoding the fragment of a GDNF precursor protein. Regulatory sequences (e.g., promoters, enhancers, polyadenylation signals, or terminators) do not typically encode a gene product, but instead affect the expression of a nucleic acid sequence. Such transformed or transfected cells can then be used, for example, for large or small scale production of the relevant fragment of a GDNF precursor protein by methods known in the art. In essence, such methods involve culturing the cells under conditions suitable for production of the fragment of a GDNF precursor protein (or the biologically active variant thereof) and isolating the fragment of a GDNF precursor protein from the cells or from the culture medium.

A construct can include a tag sequence designed to facilitate subsequent manipulations of the expressed nucleic acid sequence (e.g., purification or localization). Tag sequences, such as green fluorescent protein (GFP), glutathione S-transferase (GST), c-myc, hemagglutinin, β-galactosidase, or Flag™ tag (Kodak) sequences are typically expressed as a fusion with the polypeptide encoded by the nucleic acid sequence. Such tags can be inserted in a nucleic acid sequence such that they are expressed anywhere along an encoded polypeptide including, for example, at either the carboxyl or amino termini. The type and combination of regulatory and tag sequences can vary with each particular host, cloning or expression system, and desired outcome. A variety of cloning and expression vectors containing combinations of regulatory and tag sequences are commercially available. Suitable cloning vectors include, without limitation, pUC18, pUC19, and pBR322 and derivatives thereof (New England Biolabs, Beverly, Mass.), and pGEN (Promega, Madison, Wis.). Additionally, representative prokaryotic expression vectors include, without limitation, pBAD (Invitrogen, Carlsbad, Calif.), the pTYB family of vectors (New England Biolabs), and pGEMEX vectors (Promega); representative mammalian expression vectors include, without limitation, pTet-On/pTet-Off (Clontech, Palo Alto, Calif.), pIND, pVAX1, pCR3.1, pcDNA3.1, pcDNA4, or pUni (Invitrogen), and pCI or pSI (Promega); representative insect expression vectors include, without limitation, pBacPAK8 or pBacPAK9 (Clontech), and p2Bac (Invitrogen); and representative yeast expression vectors include, without limitation, MATCHMAKER (Clontech) and pPICZ A, B, and C (Invitrogen).

In bacterial systems, Escherichia coli can be used to express fragments of a GDNF precursor protein or biologically active variants thereof. For example, the E. coli strain DH10B (Invitrogen) can be transformed with the gram negative broad host range vector, pCM66 containing a nucleic acid sequence encoding a fragment of a GDNF precursor protein. In another example, BL-21 cells can be transformed with a pGEX vector containing a nucleic acid sequence encoding a fragment of a GDNF precursor protein. The transformed bacteria can be grown exponentially and then stimulated with isopropylthiogalactopyranoside (IPTG) prior to harvesting. In general, the fragment of a GDNF precursor protein-GST fusion polypeptides produced from a pGEX expression vector can be purified from lysed cells by adsorption to glutathione-agarose beads followed by elution in the presence of free glutathione. The pGEX vectors can be designed to include thrombin or factor Xa protease cleavage sites so that the expressed fragment of a GDNF precursor protein can be released from the GST moiety.

The invention further encompasses peptidomimetics of fragments of a GDNF precursor protein, which are small, protein-like polymers containing non-peptidic structural elements that are capable of mimicking or antagonizing the biological actions of a natural parent peptide (here, a fragment of a GDNF precursor protein). In addition to being synthetic, non-peptide compounds, peptidomimetics have a three-dimensional conformation (i.e., a “peptide motif”) that is substantially the same as the three-dimensional conformation of a selected peptide. The peptide motif provides the peptidomimetic compound with the ability to bind the receptor in a manner qualitatively identical to that of the parent peptide from which the peptiomimetic was derived. Peptidomimetic compounds can have additional characteristics that enhance their therapeutic utility, such as an increased biological half-life.

The peptidomimetics typically have a backbone that is partially or completely non-peptide, but with side groups that are identical to the side groups of the amino acid residues that occur in the peptide on which the peptidomimetic is based. Several types of chemical bonds (e.g., ester, thioester, thioamide, retroamide, reduced carbonyl, dimethylene and ketomethylene bonds) are known in the art to be generally useful substitutes for peptide bonds in the construction of protease-resistant peptidomimetics and can be used in the context of the present peptides).

Any peptidomimetic that has a sufficient amount of biological activity (e.g., an amount that renders the peptide experimentally or clinically useful in lieu of GDNF or a fragment of a GDNF precursor protein) can be used.

Biologically active variants of fragments of a GDNF precursor protein can include structural modifications. For example, one can chemically modify the peptide backbone and/or one or more side chains. Chemical modifications can be naturally occurring modifications made in vivo following translation of an mRNA encoding the polypeptide (e.g., glycosylation in a bacterial host) or synthetic modifications made in vitro. A biologically active variant of a fragment of a GDNF precursor protein can include one or more structural modifications resulting from any combination of naturally occurring (i.e., made naturally in vivo) and synthetic modifications (i.e., naturally occurring or non-naturally occurring modifications made in vitro). Examples of modifications include, but are not limited to, amidation (e.g., replacement of the free carboxyl group at the C-terminus by an amino group); biotinylation (e.g., acylation of lysine or other reactive amino acid residues with a biotin molecule); glycosylation (e.g., addition of a glycosyl group to either asparagines, hydroxylysine, serine or threonine residues to generate a glycoprotein or glycopeptide); acetylation (e.g., the addition of an acetyl group, typically at the N-terminus of a polypeptide); alkylation (e.g., the addition of an alkyl group); isoprenylation (e.g., the addition of an isoprenoid group); lipoylation (e.g. attachment of a lipoate moiety); and phosphorylation (e.g., addition of a phosphate group to serine, tyrosine, threonine or histidine).

A particularly suitable post-translational modification for fragments of a GDNF precursor protein is amidation. In vivo, amidation typically occurs at internal glycine residues and requires the sequential actions of three enzymes: two proteases (“paired basics”-specific endopetidase (e.g., prohormone convertase I) and carboxypetidase H) that cleave the precursor at the glycine residues and the amidating enzyme, peptidylglycine amidating monooxygenase (PAM). PAM catalyzes amide formation by hydroxylation of the glycine residue; the hydroxyglycine derivative dissociates to form a peptide that includes a C-terminal amide and glyoxylic acid. Methods for chemical synthesis of peptides amidated at the C-terminus are well known in the art. The synthesis can be carried out in solution or by solid-phase peptide synthetic techniques. Specific solid-phase methods for generating the amide group include, for example, without limitation, acidolysis of a benzhydral amide linkage between the peptide and the solid-support and ammonolysis of a peptide-resin ester linkage.

As noted above in describing suitable expression vectors, the present peptides can include a tag, which may also be referred to as a reporter or marker (e.g., a detectable marker). A detectable marker can be any molecule that is covalently linked to the fragment of a GDNF precursor protein or a biologically active fragment thereof that allows for qualitative and/or quantitative assessment of the expression or activity of the tagged peptide. The activity can include a biological activity, a physico-chemical activity, or a combination thereof. Both the form and position of the detectable marker can vary, as long as the labeled peptide retains biological activity. Many different markers can be used, and the choice of a particular marker will depend upon the desired application. Labeled fragments of a GDNF precursor protein can be used, for example, for anatomical or cytochemical mapping of GDNF receptor distribution in order to identify populations of target cells for a particular peptide or for evaluating phamacokinetics of a fragment of a GDNF precursor protein both in cell-based systems and in whole animal models.

Suitable markers include, for example, enzymes, photo-affinity ligands, radioisotopes, and fluorescent or chemiluminescent compounds. Methods of introducing detectable markers into peptides are well known in the art. Markers can be added during synthesis or post-synthetically. Recombinant fragments of a GDNF precursor protein or biologically active variants thereof can also be labeled by the addition of labeled precursors (e.g., radiolabeled amino acids) to the culture medium in which the transformed cells are grown. In some embodiments, analogues or variants of peptides can be used in order to facilitate incorporation of detectable markers. For example, any N-terminal phenylalanine residue can be replaced with a closely related aromatic amino acid, such as tyrosine, that can be easily labeled with ¹²⁵I. In some embodiments, additional functional groups that support effective labeling can be added to the fragments of a GDNF precursor protein or biologically active variants thereof. For example, a 3-tributyltinbenzoyl group can be added to the N-terminus of the native structure; subsequent displacement of the tributyltin group with ¹²⁵I will generate a radiolabeled iodobenzoyl group.

The fragments of a GDNF precursor protein can also include a detectable marker that is transferred to the GDNF receptor upon peptide-receptor binding. For example, phytochemical activation of a peptide analog that includes an N-terminal 4-azidobenzoyl group and an iodotyrosine residue in place of the phenylalanine residue could be used to covalently radiolabel GDNF receptors.

Nucleic Acids

The terms “nucleic acid” and “polynucleotide” are used interchangeably herein, and refer to both RNA and DNA, including cDNA, genomic DNA, synthetic DNA, and DNA (or RNA) containing nucleic acid analogs. Polynucleotides can have any three-dimensional structure. A nucleic acid can be double-stranded or single-stranded (i.e., a sense strand or an antisense strand). Non-limiting examples of polynucleotides include genes, gene fragments, exons, introns, messenger RNA (mRNA) and portions thereof, transfer RNA, ribosomal RNA, siRNA, micro-RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs. In the context of the present invention, nucleic acids can encode a fragment of a GDNF precursor protein or a biologically active fragment thereof.

An “isolated” nucleic acid can be, for example, a naturally-occurring DNA molecule or a fragment thereof, provided that at least one of the nucleic acid sequences normally found immediately flanking that DNA molecule in a naturally-occurring genome is removed or absent. Thus, an isolated nucleic acid includes, without limitation, a DNA molecule that exists as a separate molecule, independent of other sequences (e.g., a chemically synthesized nucleic acid, or a cDNA or genomic DNA fragment produced by the polymerase chain reaction (PCR) or restriction endonuclease treatment). An isolated nucleic acid also refers to a DNA molecule that is incorporated into a vector, an autonomously replicating plasmid, a virus, or into the genomic DNA of a prokaryote or eukaryote. In addition, an isolated nucleic acid can include an engineered nucleic acid such as a DNA molecule that is part of a hybrid or fusion nucleic acid. A nucleic acid existing among many (e.g., dozens, or hundreds to millions) of other nucleic acids within, for example, cDNA libraries or genomic libraries, or gel slices containing a genomic DNA restriction digest, is not an isolated nucleic acid.

Isolated nucleic acid molecules can be produced by standard techniques. For example, polymerase chain reaction (PCR) techniques can be used to obtain an isolated nucleic acid containing a nucleotide sequence described herein, including nucleotide sequences encoding a polypeptide described herein (i.e. a fragment of a GDNF precursor protein or a biologically active variant thereof). PCR can be used to amplify specific sequences from DNA as well as RNA, including sequences from total genomic DNA or total cellular RNA. Various PCR methods are described in, for example, PCR Primer: A Laboratory Manual, Dieffenbach and Dveksler, eds., Cold Spring Harbor Laboratory Press, 1995. Generally, sequence information from the ends of the region of interest or beyond is employed to design oligonucleotide primers that are identical or similar in sequence to opposite strands of the template to be amplified. Various PCR strategies also are available by which site-specific nucleotide sequence modifications can be introduced into a template nucleic acid (as one may wish to do, for example, when making a biologically active variant of a fragment of a GDNF precursor protein).

Isolated nucleic acids also can be chemically synthesized, either as a single nucleic acid molecule (e.g., using automated DNA synthesis in the 3′ to 5′ direction using phosphoramidite technology) or as a series of oligonucleotides. For example, one or more pairs of long oligonucleotides (e.g., >50-100 nucleotides) can be synthesized that contain the desired sequence, with each pair containing a short segment of complementarity (e.g., about 15 nucleotides) such that a duplex is formed when the oligonucleotide pair is annealed. DNA polymerase is used to extend the oligonucleotides, resulting in a single, double-stranded nucleic acid molecule per oligonucleotide pair, which then can be ligated into a vector. Isolated nucleic acids of the invention also can be obtained by mutagenesis of, e.g., a naturally occurring portion of a GDNF-encoding DNA (in accordance with, for example, one of Formulas I, II, or III).

Two nucleic acids or the polypeptides they encode may be described as having a certain degree of identity to one another. For example, a fragment of a GDNF precursor protein and a biologically active variant thereof may be described as exhibiting a certain degree of identity. Alignments may be assembled by locating short GDNF sequences in the Protein Information Research (PIR) site. (http://pir.georgetown.edu) followed by analysis with the “short nearly identical sequences” Basic Local Alignment Search Tool (BLAST) algorithm on the NCBI website (http://www.ncbi.nlm.nih.gov/blast).

As used herein, the term “percent sequence identity” refers to the degree of identity between any given query sequence and a subject sequence. For example, a naturally occurring GDNF can be the query sequence and a fragment of a GDNF precursor protein can be the subject sequence. Similarly, a fragment of a GDNF precursor protein can be the query sequence and a biologically active variant thereof can be the subject sequence.

To determine sequence identity, a query nucleic acid or amino acid sequence can be aligned to one or more subject nucleic acid or amino acid sequences, respectively, using the computer program ClustalW (version 1.83, default parameters), which allows alignments of nucleic acid or protein sequences to be carried out across their entire length (global alignment). See Chema et al., Nucleic Acids Res. 31:3497-3500, 2003.

ClustalW calculates the best match between a query and one or more subject sequences and aligns them so that identities, similarities and differences can be determined. Gaps of one or more residues can be inserted into a query sequence, a subject sequence, or both, to maximize sequence alignments. For fast pairwise alignment of nucleic acid sequences, the following default parameters are used: word size: 2; window size: 4; scoring method: percentage; number of top diagonals: 4; and gap penalty: 5. For multiple alignment of nucleic acid sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gln, Glu, Arg, and Lys; residue-specific gap penalties: on. The output is a sequence alignment that reflects the relationship between sequences. ClustalW can be run, for example, at the Baylor College of Medicine Search Launcher site (searchlauncher.bcm.tmc.edu/multi-align/multi-align.html) and at the European Bioinformatics Institute site on the World Wide Web (ebi.ac.uk/clustalw).

To determine a percent identity between a query sequence and a subject sequence, ClustalW divides the number of identities in the best alignment by the number of residues compared (gap positions are excluded), and multiplies the result by 100. The output is the percent identity of the subject sequence with respect to the query sequence. It is noted that the percent identity value can be rounded to the nearest tenth. For example, 78.11, 78.12, 78.13, and 78.14 are rounded down to 78.1, while 78.15, 78.16, 78.17, 78.18, and 78.19 are rounded up to 78.2.

The nucleic acids and polypeptides described herein may be referred to as “exogenous”. The term “exogenous” indicates that the nucleic acid or polypeptide is part of, or encoded by, a recombinant nucleic acid construct, or is not in its natural environment. For example, an exogenous nucleic acid can be a sequence from one species introduced into another species, i.e., a heterologous nucleic acid. Typically, such an exogenous nucleic acid is introduced into the other species via a recombinant nucleic acid construct. An exogenous nucleic acid can also be a sequence that is native to an organism and that has been reintroduced into cells of that organism. An exogenous nucleic acid that includes a native sequence can often be distinguished from the naturally occurring sequence by the presence of non-natural sequences linked to the exogenous nucleic acid, e.g., non-native regulatory sequences flanking a native sequence in a recombinant nucleic acid construct. In addition, stably transformed exogenous nucleic acids typically are integrated at positions other than the position where the native sequence is found.

Recombinant constructs are also provided herein and can be used to transform cells in order to express fragments of a GDNF precursor protein. A recombinant nucleic acid construct comprises a nucleic acid encoding a fragment of a GDNF precursor protein as described herein, operably linked to a regulatory region suitable for expressing the fragment of a GDNF precursor protein in the cell. Thus, a nucleic acid can comprise a coding sequence that encodes any of the fragments of a GDNF precursor protein as set forth in SEQ ID NOs: 1, 2, and 3 and the consensus sequences set forth herein (e.g., in FIG. 2). In some cases, a recombinant nucleic acid construct can include a nucleic acid comprising a coding sequence, a gene, or a fragment of a coding sequence or gene in an antisense orientation so that the antisense strand of RNA is transcribed. It will be appreciated that a number of nucleic acids can encode a polypeptide having a particular amino acid sequence. The degeneracy of the genetic code is well known in the art. For many amino acids, there is more than one nucleotide triplet that serves as the codon for the amino acid. For example, codons in the coding sequence for a given fragment of a GDNF precursor protein can be modified such that optimal expression in a particular organism is obtained, using appropriate codon bias tables for that organism.

Vectors containing nucleic acids such as those described herein also are provided. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes a regulatory region. Suitable expression vectors include, without limitation, plasmids and viral vectors derived from, for example, bacteriophage, baculoviruses, and retroviruses. Numerous vectors and expression systems are commercially available from such corporations as Novagen (Madison, Wis.), Clontech (Palo Alto, Calif.), Stratagene (La Jolla, Calif.), and Invitrogen/Life Technologies (Carlsbad, Calif.).

The vectors provided herein also can include, for example, origins of replication, scaffold attachment regions (SARs), and/or markers. A marker gene can confer a selectable phenotype on a host cell. For example, a marker can confer biocide resistance, such as resistance to an antibiotic (e.g., kanamycin, G418, bleomycin, or hygromycin). As noted above, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

The vector can also include a regulatory region. The term “regulatory region” refers to nucleotide sequences that influence transcription or translation initiation and rate, and stability and/or mobility of a transcription or translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, and introns.

As used herein, the term “operably linked” refers to positioning of a regulatory region and a sequence to be transcribed in a nucleic acid so as to influence transcription or translation of such a sequence. For example, to bring a coding sequence under the control of a promoter, the translation initiation site of the translational reading frame of the polypeptide is typically positioned between one and about fifty nucleotides downstream of the promoter. A promoter can, however, be positioned as much as about 5,000 nucleotides upstream of the translation initiation site or about 2,000 nucleotides upstream of the transcription start site. A promoter typically comprises at least a core (basal) promoter. A promoter also may include at least one control element, such as an enhancer sequence, an upstream element or an upstream activation region (UAR). The choice of promoters to be included depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning promoters and other regulatory regions relative to the coding sequence.

Methods of Treatment

The fragments of a GDNF precursor protein disclosed herein and biologically active variants thereof are useful for the treatment of neurological disorders, including those amenable to treatment with wildtype GDNF. Treatment can completely or partially abolish some or all of the signs and symptoms of the neurological disorder, decrease the severity of the symptoms, delay their onset, or lessen the progression or severity of subsequently developed symptoms.

Neurological disorders that can be treated with the peptides described herein include those in which either motor or sensory neurons die or dysfunction. These disorders may be the result of disease, injury, or of an unknown cause, and they may be influenced by one's genetic constitution. Neuronal dysfunction and/or degeneration are common underlying features of many neurological disorders. A patient who has a neurological disorder associated with dysfunctional or degenerating GDNF-responsive neurons is a candidate for treatment with the peptides described herein.

Motor system disorders amenable to treatment may be characterized by any combination of atypical movement such as tremor, trembling of the hands, arms, legs, jaw, and face; rigidity, or stiffness of the limbs and trunk; bradykinesia; postural instability; impaired balance and coordination; muscular rigidity; involuntary or nonrepetitive and occasionally stereotypical movements; and ataxia.

The symptoms described above can be associated with a loss of, or damage to, particular neurons in the brain, including, but not limited to: dopaminergic neurons, the pigmented neurons of the substantia nigra, the locus caeruleus, brain stem dopaminergic cells, the basal ganglia, the archicerebellum, the vestibulocerebellum and the neocerebellum. Examples of motor system disorders include, but are not limited to, Parkinson's disease, ataxia or dyskinesia of unknown etiology, multiple system atrophy, Shy-Drager syndrome, progressive supranuclear palsy, essential tremor, Friedreich's ataxia, and cerebellar ataxia. The present peptides are applicable to motor system disorders arising from idiopathic or genetic causes (e.g., primary Parkinsonism), as well as motor system disorders such as secondary Parkinsonism that result from trauma or drug use. More specifically, secondary Parkinsonism can result from abuse of prescription (e.g., antipsychotic) or recreational drugs; abuse of alcohol; exposure to a toxin (e.g., carbon monoxide poisoning); structural lesions (e.g., tumors or infarcts affecting the midbrain or basal ganglia); or as a sequella to infectious disease, e.g., postencephalitic Parkinsonism.

The peptides provided herein are also applicable to conditions resulting from disorders of sensory neurons, including those involved in hearing. Sensorineural hearing loss is caused by malfunction of the inner ear (cochlea, cochlea hair cells, eighth cranial nerve) or auditory brainstem. Sensorineural hearing loss can result from many factors including genetic factors, trauma, disease or aging.

In another embodiment, the present peptides can be used to treat neurologic syndromes such as attention deficit disorders (e.g., ADHD) and drug addiction. Attention-deficit/hyperactivity disorder (ADHD) (formerly known as ADD) is usually diagnosed in childhood. Symptoms of ADHD can include hyperactivity, forgetfulness, poor impulse control, and distractibility. Other neurological disorders encompassed by the invention include Alzheimer's disease and Huntington's disease.

With respect to addiction, the present peptides can be used to treat addiction to a variety of substances or activities. For example, the compositions can be used to treat addiction to stimulants (e.g., cocaine, amphetamines, methamphetamines, methylphenidate, and related stimulants), opiates (e.g., heroin, codeine, hydrocodone, and related opioid drugs), nicotine, alcohol, prescription medications (e.g., medications prescribed for pain management such as Percodan™ or Percocet™), and naturally-occurring plant-derived drugs (e.g. marijuana). Patients being treated with methadone are also candidates for treatment with the compositions described herein. The present compositions may help such patients step-down and discontinue use of methadone. Addictive behaviors can also be directed toward activities including addictions to eating, gambling, or sexual behavior.

Administration and Formulation

Fragments of a GDNF precursor protein and biologically active variants thereof can be administered directly to a mammal. Generally, the peptides can be suspended in a pharmaceutically acceptable carrier (e.g., physiological saline or a buffered saline solution) to facilitate their delivery. Encapsulation of the polypeptides in a suitable delivery vehicle (e.g., polymeric microparticles or implantable devices) may increase the efficiency of delivery. A composition can be made by combining any of the peptides provided herein with a pharmaceutically acceptable carrier. Such carriers can include, without limitation, sterile aqueous or non-aqueous solutions, suspensions, and emulsions. Examples of non-aqueous solvents include mineral oil, propylene glycol, polyethylene glycol, vegetable oils, and injectable organic esters. Aqueous carriers include, without limitation, water, alcohol, saline, and buffered solutions. Preservatives, flavorings, and other additives such as, for example, antimicrobials, anti-oxidants (e.g., propyl gallate), chelating agents, inert gases, and the like may also be present. It will be appreciated that any material described herein that is to be administered to a mammal can contain one or more pharmaceutically acceptable carriers.

Any composition described herein can be administered to any part of the host's body for subsequent delivery to a GDNF-responsive cell. A composition can be delivered to, without limitation, the brain, the cerebrospinal fluid, joints, nasal mucosa, blood, lungs, intestines, muscle tissues, skin, or the peritoneal cavity of a mammal. In terms of routes of delivery, a composition can be administered by intravenous, intracranial, intraperitoneal, intramuscular, subcutaneous, intramuscular, intrarectal, intravaginal, intrathecal, intratracheal, intradermal, or transdermal injection, by oral or nasal administration, or by gradual perfusion over time. In a further example, an aerosol preparation of a composition can be given to a host by inhalation.

The dosage required will depend on the route of administration, the nature of the formulation, the nature of the patient's illness, the patient's size, weight, surface area, age, and sex, other drugs being administered, and the judgment of the attending clinician. Suitable dosages are in the range of 0.01-1,000 μg/kg, for example 0.01-1 μg/kg; 0.05-10 μg/kg; 5-100 μg/kg; 50-200 μg/kg; 150-300 μg/kg; 200-500 μg/kg; 300-600 μg/kg; 400-800 μg/kg; 500-900 μg/kg; 600-1000 μg/kg. Wide variations in the needed dosage are to be expected in view of the variety of fragments of a GDNF precursor protein and biologically active variants available and the differing efficiencies of various routes of administration. Variations in these dosage levels can be adjusted using standard empirical routines for optimization, as is well understood in the art. Administrations can be single or multiple (e.g., 2- or 3-, 4-, 6-, 8-, 10-, 20-, 50-, 100-, 150-, or more fold). Encapsulation of the fragments of a GDNF precursor protein in a suitable delivery vehicle (e.g., polymeric microparticles or implantable devices) may increase the efficiency of delivery.

The duration of treatment with any composition provided herein can be any length of time from as short as one day to as long as the life span of the host (e.g., many years). For example, fragments of a GDNF precursor protein and biologically active variants thereof can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present peptides can be administered once (or twice, three times, etc.) daily, weekly, monthly, or yearly.

An effective amount of any composition provided herein can be administered to an individual in need of treatment. The term “effective” as used herein refers to any amount that induces a desired response while not inducing significant toxicity in the patient. Such an amount can be determined by assessing a patient's response after administration of a known amount of a particular composition. In addition, the level of toxicity, if any, can be determined by assessing a patient's clinical symptoms before and after administering a known amount of a particular composition. It is noted that the effective amount of a particular composition administered to a patient can be adjusted according to a desired outcome as well as the patient's response and level of toxicity. Significant toxicity can vary for each particular patient and depends on multiple factors including, without limitation, the patient's disease state, age, and tolerance to side effects.

Any method known to those in the art can be used to determine if a particular response is induced. Clinical methods that can assess the degree of a particular disease state can be used to determine if a response is induced. For example, in a Parkinson's disease patient, a lessening in spontaneous movements, an improvement in gait and posture, and a reduction in the severity of tremors can be indicative of a neurological response in a patient treated with the present peptides. For some disorders, blood or laboratory tests, or psychological evaluations can be used to assist the clinician in evaluating a patient's response to fragments of a GDNF precursor protein. The particular methods used to evaluate a response will depend upon the nature of the patient's disorder, the patient's age, and sex, other drugs being administered, and the judgment of the attending clinician.

Alternatively, a polynucleotide containing a nucleic acid sequence encoding a fragment of a GDNF precursor protein or a biologically active fragment thereof can be delivered to an appropriate cell of the animal. This can be achieved by, for example, the use of a polymeric, biodegradable microparticle or microcapsule delivery vehicle, sized to optimize phagocytosis by phagocytic cells such as macrophages. For example, PLGA (poly-lactide-co-glycolide) microparticles approximately 1-10 μm in diameter can be used. The polynucleotide is encapsulated in these microparticles, which are taken up by macrophages and gradually biodegraded within the cell, thereby releasing the polynucleotide. Once released, the DNA is expressed within the cell. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm).

Another way to achieve uptake of the nucleic acid is using liposomes, prepared by standard methods. The vectors can be incorporated alone into these delivery vehicles or co-incorporated with tissue-specific antibodies. Alternatively, one can prepare a molecular conjugate composed of a plasmid or other vector attached to poly-L-lysine by electrostatic or covalent forces. Poly-L-lysine binds to a ligand that can bind to a receptor on target cells. Delivery of “naked DNA” (i.e., without a delivery vehicle) to an intramuscular, intradermal, or subcutaneous site, is another means to achieve in vivo expression.

In the relevant polynucleotides (e.g., expression vectors) the nucleic acid sequence encoding the fragment of a GDNF precursor protein of interest (or the biologically active variant thereof) with an initiator methionine and optionally a targeting sequence is operatively linked to a promoter or enhancer-promoter combination. Promoters and enhancers are described above, and many are well known in the art.

Polynucleotides can be administered in a pharmaceutically acceptable carrier. Pharmaceutically acceptable carriers are biologically compatible vehicles which are suitable for administration to a human or other mammalian subject (e.g., physiological saline). A therapeutically effective amount is an amount of the polynucleotide which is capable of producing a medically desirable result (e.g., a decrease in clinical motor symptoms) in a treated mammal. As is well known in the medical arts, the dosage for any one patient depends upon many factors, including the patient's size, body surface area, age, the particular compound to be administered, sex, time and route of administration, general health, and other drugs being administered concurrently. Dosages will vary, but a preferred dosage for administration of polynucleotide is from approximately 10⁶ to 10¹² copies of the polynucleotide molecule. This dose can be repeatedly administered, as needed. Routes of administration can be any of those listed above.

The fragments of a GDNF precursor protein disclosed herein can be administered as a single fragment of a GDNF precursor protein or a biologically active variant thereof (e.g., a fragment of a GDNF precursor protein of Formula I, Formula II, or Formula III). The formulation of GDNF-peptides can include a mixture of peptides that conform to the wild-type GNDF amino acid sequence with fragments of a GDNF precursor protein that are biologically active variants of the wild type sequence. For example, formulations of fragments of a GDNF precursor protein can be formulated as a combination of peptides of 2, 3, 4, 5, 6, 7, 8, 9, 10, 15, 10, 30, or more different amino acid sequences. Thus, for example, the compositions can include mixtures of polypeptides conforming to Formula I; mixtures of polypeptides conforming to Formula II; mixtures of polypeptides conforming to Formula III; mixtures of polypeptides conforming to Formulas I and II; mixtures of polypeptides conforming to Formulas II and III; mixtures of polypeptides conforming to Formulas I and III; or mixtures of polypeptides conforming to Formulas I, II, and III. The formulation can also include a mixture of GNDF-derived peptides, one or more of which have been post-synthetically modified (e.g., amidated fragments of a GDNF precursor protein with unmodified fragments of a GDNF precursor protein). Where nucleic acids are formulated as pharmaceutical compositions, the nucleic acids can similarly encode polypeptides in the configurations just described.

The peptides provided herein can be administered in conjunction with other therapeutic modalities to an individual in need of therapy. The fragments of a GDNF precursor protein and biologically active variants thereof can be given prior to, simultaneously with or after treatment with other agents or regimes (e.g., a radiotherapy regime).

The fragments of a GDNF precursor protein can be administered in conjunction with other therapies for treating neurological disorders, such as standard, small molecule-type pharmaceutical agents, biopharmaceuticals (e.g., antibodies or antibody-related immunotherapies, siRNAs, shRNAs, antisense oligonucleotides and other RNA inhibitory molecules, microRNAs, and peptide therapeutics), surgery, or in conjuction with any medical devices that may be used to assist the patient. Standard therapies for treating neurological disorders can include, for example, levodopa, carbidopa, anticholinergics, dopamine mimics such as bromocriptine, pergolide, pramipexole, and ropinirole, monoamine oxidase (MAO) type-B inhibitors, such as rasagiline and its analogues, and antiviral drugs, such as amantidine, acetylcholinesterase inhibitors such as donepezil, galantamine, and memantine, as well as surgical remedies such as deep brain stimulation. The fragments of a GDNF precursor protein can be administered in conjunction with standard agents for treating ADHD, e.g., methylphenidate (Ritalin) or dextroamphetamine (Dexedrine). The fragments of a GDNF precursor protein can be administered in conjunction with standard agents for treating addiction e.g., methadone.

The fragments of a GDNF precursor protein can be also administered in conjunction with other standard remedies such as surgery or the use of medical devices, for example hearing aids.

Kits

The compositions described herein can also be assembled in kits, together with instructions for use. For example, the kits can include measured amounts of a pharmaceutically acceptable composition including fragments of a GDNF precursor protein and/or biologically active variants thereof. The instructions for use can be conveyed by any suitable media. For example, they can be printed on a paper insert in one or more languages or supplied audibly or visually (e.g., on a compact disc). The packaging materials can include vials, packets, or intravenous bags, and the kit can also include instruments useful in administration, such as needles, syringes, tubing, catheters, bandages, and tape. Preferably, the components of the kit are sterile and suitable for immediate use. The invention encompasses kits, however, that included concentrated formulations and/or materials that may require sterilization prior to use.

### EXAMPLES

### Example 1

### GDNF Peptide Sequences

The amino acid sequence of human GDNF is shown in FIG. 1. The locations of the relevant fragments of GDNF precursor protein are italicized and/or underlined as indicated. The sequences of individual fragments are presented in Table 1. Potential amidation signals are underlined.

TABLE 1
GDNF peptide sequences

| | | SEQ |
|---|---|---|
| | | ID |
| GDNF peptide | Sequence | NO: |
| GDNF peptide I | FPLPAGKR | 1 |
| GDNF peptide II | PPEAPAEDRSLGRR | 2 |
| GDNF peptide III | RRERNRQAAAANPENSRGKGRR | 3 |

### Example 2

### Sequence Conservation of GDNF Peptides

Homologous sequences from other species were identified using the “short nearly identical sequences” Basic Local Alignment Search Tool (BLAST) algorithm on the NCBI website (http://www.ncbi.nlm.nih.gov/blast) and the Protein Information Research (PIR) site (http://www.pir.georgetown.edu). Amino acid sequence alignments of the GDNF homologues of specific peptides are shown in FIG. 2. The Formula I sequence (designated in FIG. 2 as GDNF Consensus Neuropeptide I) was invariant in the species available (Homo sapiens, R. norvegicus, and Mus musculus). The Formula II homologues (designated in FIG. 2 as GDNF Consensus Neuropeptide II) did not contain the amidation signals found in the human GDNF peptide II. Species-specific variants of Homo sapiens Formula III sequence (designated in FIG. 2 as GDNF Consensus Neuropeptide III) are shown for Macaca mulata, Rattus norvegicus, Mus musculus, Rattus specis, A. melanoleuca, Bos taurus, Gallus gallus, and Nipponia Nippon. The differential residues are underlined for ease of identification.

### Example 3

### Analysis Ca²⁺ Release in GDNF Peptide-Treated Cells

We evaluated the ability of the GDNF peptides to activate G-protein associated transmembrane receptors. C6 glial cells were incubated with GDNF peptides, either singly or in combination, and calcium mobilization was monitored as a index of receptor activation.

GDNF peptides were custom synthesized via automated solid-phase methods using FMOC chemistry, HPLC purified, and characterized at the Small Peptide Synthesis Resource of the KECK Facility at Yale University. The sequences of the peptides used in the experiments described in Examples 4-7 are provided in Table 2. C6 glial cells were cultured to 80% confluency in the presence of GDNF peptides and loaded with the Ca²⁺-sensitive fluorescent indicator dye, Calcium Crimson-AM (5 μM dye in Me₂SO) at 37° C. for 20 min. Approximately 10³ cells were then transferred into a quartz cuvette and the change in intracellular fluorescence was monitored in real time using an ISI Fluorescence spectrophotometer with an excitation wavelength of 590 nm. Emissions were monitored at 615 nm. An increase in fluorescence units was indicative of a rise in internal calcium.

### Example 4

### Effect of GDNF-Derived Peptide (FPLPA (SEQ ID NO:4)) on Ca²⁺ Flux in C6 Glial Cells

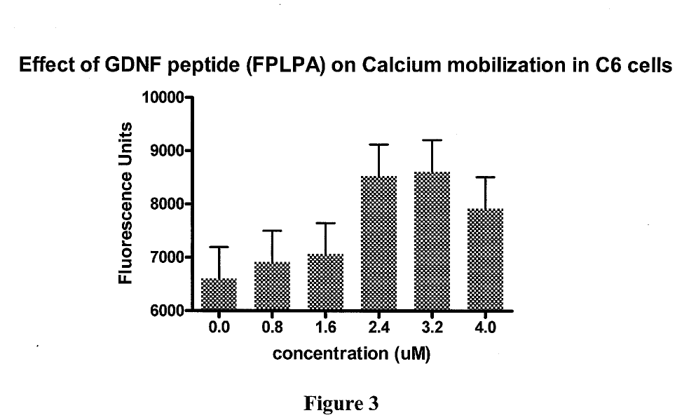

C6 glial cells were cultured as above and incubated in increasing concentrations (0.8 μM; 1.6 μM; 2.4 μM; 3.2 μM and 4.0 μM) of GDNF-derived peptide (FPLPA (SEQ ID NO:4)). Fluorescence was measured according to the method in Example 3. As shown in FIG. 3, this peptide induced a dose-dependent increase in Ca²⁺ mobilization at peptide concentrations up to 3.2 μM, followed by a decrease at 4.0 μM. These data suggest that the GDNF-derived peptide FPLPA (SEQ ID NO:4) can activate G-protein associated transmembrane receptors on neuronal cells.

### Example 5

### Effect of GDNF-Derived Peptide (PPEAPAEDRSL (SEQ ID NO:5)) on Ca²⁺ Flux in C6 Glial Cells

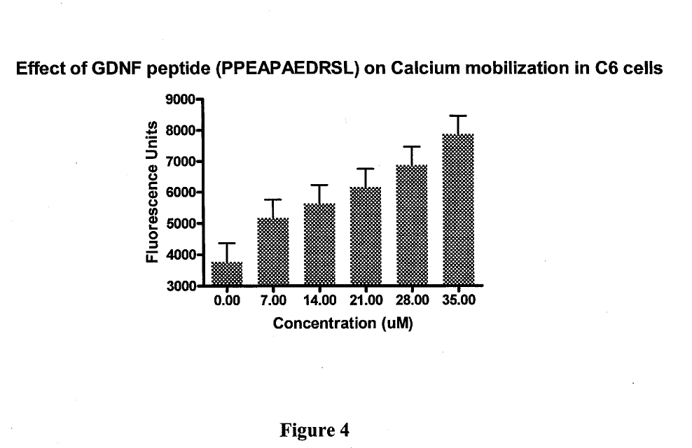

C6 glial cells were cultured as above and incubated in increasing concentrations (7.0 μM; 14 μM; 21 μM; 28 μM and 35 μM) of GDNF-derived peptide (PPEAPAEDRSL (SEQ ID NO:5)). Fluorescence was measured according to the method in Example 3. As shown in FIG. 4, GDNF-derived peptide PPEAPAEDRSL (SEQ ID NO:5) induced a dose-dependent increase in Ca²⁺ mobilization. These data suggest that the GDNF-derived peptide PPEAPAEDRSL (SEQ ID NO:5) can activate G-protein associated transmembrane receptors on neuronal cells.

### Example 6

### Effect of GDNF-Derived Peptides Individually or in Combination on Ca²⁺ Flux in C6 Glial Cells (External Ca²⁺ Free Buffer)

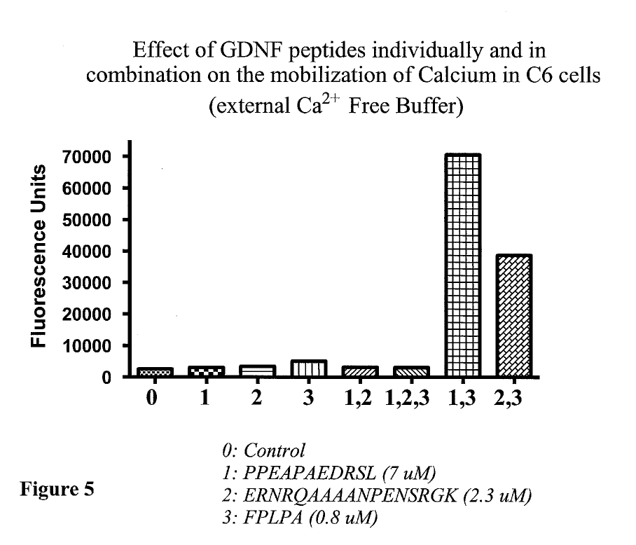

C6 glial cells were cultured as above except that no Ca²⁺ was present in the media. Cells were incubated with GDNF-derived peptides according to the experimental design indicated in Table 2 and the combinations in FIG. 5. Fluorescence was measured according to the method in Example 3.

TABLE 2
Experimentl design for Exmples 5 nd 6

| | SEQ | # in | |
|---|---|---|---|
| | ID | FIGS. 4 | Concentrtion |
| GDNF peptide sequence | NO: | nd 5 | (μM) |
| FPLPA | 4 | 3 | 0.8 |
| PPEAPAEDRSL | 5 | 1 | 7.0 |
| ERNRQAAAANPENSRGK | 6 | 2 | 2.3 |

As indicated in FIG. 5, moderate increases in fluorescence relative to the control sample were noted for the individual peptides (indicated in bars 1, 2, and 3) as well as for the combinations (1, 2) and (1, 2, 3). An increase of more than 20-fold relative to the control was observed with the combination (1, 3), a mixture of GDNF-derived peptide (PPEAPAEDRSL; SEQ ID NO:5) and GDNF-derived peptide (FPLPA; SEQ ID NO:4) respectively, and an increase of more that 10-fold relative to the control was observed with the combination (2, 3), a mixture of GDNF-derived peptide (PPEAPAEDRSL; SEQ ID NO:5) and GDNF-derived peptide (FPLPA; SEQ ID NO:4), respectively. These data suggest that specific combinations of GDNF-derived peptides have synergistic effects on activation of G-protein associated transmembrane receptors on neuronal cells.

### Example 7

### Effect of GDNF-Derived Peptides Individually or in Combination on Ca²⁺ Flux in C6 Glial Cells (External Ca²⁺ Containing Buffer)

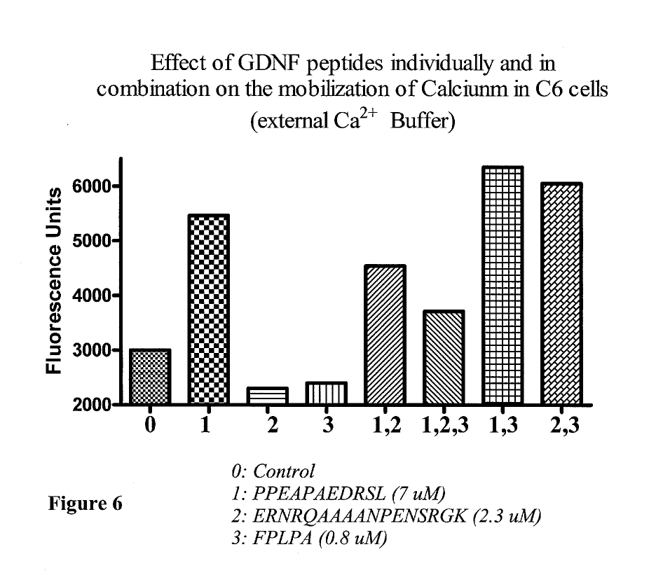

C6 glial cells were cultured according to the method in Example 3. Cells were incubated with GDNF-derived peptides according to the experimental design indicated in Table 2 and the combinations in FIG. 6. Fluorescence was measured according to the method in Example 3. As indicated in FIG. 6, GDNF-derived peptide (PPEAPAEDRSL; SEQ ID NO:5) (bar 1) stimulated an increase in Ca²⁺ mobilization relative to that of control samples; GDNF-derived peptide (ERNRQAAAANPENSRGK; SEQ ID NO:6) (bar2) and GDNF-derived peptide (FPLPA; SEQ ID NO:4) (bar 3), when administered individually produced a decrease in Ca²⁺ mobilization relative to control samples. All combinations of peptides (1,2), (1, 2, 3), (1, 3) and (2,3) stimulated an increase in Ca⁺ mobilization ranging from about 0.5-fold to more than 2-fold relative to that observed with control samples. These data suggest that specific combinations of GDNF-derived peptides have synergistic effects on activation of G-protein associated transmembrane receptors on neuronal cells.

### Example 8

### Representative GDNF cDNA Sequence

A representative cDNA sequence (NM_{—}000514.2 GI:40549401) (SEQ ID NO:7) encoding a GDNF polypeptide is shown below:

```
    1 ccgcctccg cgcgcccttg ctgccccgcg cgccccgg ttgcgct cttgcccctg
  61 cctgttggg cggggctccg cgctccgcc tcgcccgg tgggtctcc tggctgggc
121 ttggggccc tgggttt gtcccct gggtctgcgg gcccgtc cgggtgccg
181 ccgccggcg ggctttg tggttt gggtgtcgt ggctgtctgc ctggtgctgc
241 tcccccgc gtccgccttc ccgctgcccg ccggtgg gcctcccgg gcgcccgccg
301 gccgctc cctcggccgc cgccgcgcgc ccttcgcgct ggcgtgc tcttgc
361 cgggtt tcctgtcg ttcgtgtg tctggttt tttcgcc cctt
421 gctgg gtcccgt ctgg cgtgcttcc tgggg cggtcggc
481 ggctgcgc tgccccc ggttcc ggggg tcgggggc cgggggc
541 ccgggg ttgtgtctt ctgctc ttttgt cctgcttg ggtctgggct
601 tgcc ggggctg tttttggt ctgcgcgg ctcttgcgt gcgctgg
661 ccgtcg ctttg cttt ccgtg ggctggtg gtgcg
721 tgggcggc tgttgcg ccctcgcct ttgtgtg cctgtcgttt ttgtgt
781 cctggttt cctttct ggctt ccgctg gtgtggtgt tctg
```

### Example 9

### Representative GDNF Amino Acid Sequence

A representative amino acid sequence (Genbank number NP_{—}000505.1, GI:4503975) (SEQ ID NO:8) encoding a GDNF polypeptide is shown below:

```
    1 mklwdvvvc lvllhtsf plpgkrppe pedrslgr rrpflssd snmpedypdq
  61 fddvmdfiq tikrlkrspd kqmvlprre rnrqnp ensrgkgrrg qrgknrgcvl
121 tihlnvtdl glgyetkeel ifrycsgscd ettydkil knlsrnrrlv sdkvgqccr
181 pifdddlsf lddnlvyhil rkhskrcgc i
```

### Example 10

### Antigenicity of GDNF-Derived Peptides

The relative antigenicity of GDNF-derived peptides was analyzed by the method of Kolaskar and Tongaonkar (FEBS Lett. 1990; 276: 172-4). This bioinformatics tool relies on physicochemical properties of amino acid residues and their frequencies of occurrence in experimentally known segmental epitopes to predict antigenic determinants on proteins. For any given peptide, an antigenic propensity score of about 1 or less suggests that such a peptide is not antigenic. The GDNF-derived peptides analyzed were the 5-mer, FPLPA (SEQ ID NO:4); the 11-mer, PPEAPAEDRSL (SEQ ID NO:5) and the 17-mer, ERNRQAAAANPENSRGK (SEQ ID NO:6). The antigenic propensity scores were 0.9773, and 0.9294, respectively, for PPEAPAEDRSL (SEQ ID NO:5) and ERNRQAAAANPENSRGK (SEQ ID NO:6). The peptide FPLPA (SEQ ID NO:4), which was slightly below the minimum length of 8 residues for the algorithm, scored 1.1173. The results suggest that the GDNF-derived peptides may be unlikely to provoke an immune response when administered to a subject.

A number of embodiments of the invention have been described. Nevertheless, it will be understood that various modifications may be made without departing from the spirit and scope of the invention. Accordingly, other embodiments are within the scope of the following claims.

1. A substantially pure polypeptide comprising a fragment of a glial-derived neurotrophic factor (GDNF) precursor protein or a biologically active variant thereof, wherein the fragment or the variant thereof consists of not more than 92 amino acid residues and wherein the polypeptide, when administered in a therapeutically effective amount to a patient having a neurological disorder, improves a sign or symptom of the neurological disorder.

2. A substantially pure polypeptide comprising a fragment of a GDNF precursor protein or a biologically active variant thereof, wherein the fragment or the variant thereof excludes amino acid residues 118-211 of SEQ ID NO:8 and wherein the polypeptide, when administered in a therapeutically effective amount to a patient having a neurological disorder, improves a sign or symptom of the neurological disorder.

3. The polypeptide of claim 1, wherein the fragment of GDNF precursor protein or the biologically active variant thereof comprises an amino acid sequence of Formula I:
Xaa₁-Pro-Xaa₃-Pro-Xaa₅-Xaa₆-Xaa₇-Xaa₈ (I) wherein
Xaa₁ is Phe, Trp, or Tyr;
Xaa₃ is Leu, Ala, Ile, or Val;
Xaa₅ is Ala, Leu, Ile, or Val;
Xaa₆ is Gly, is any amino acid residue of the D configuration or is absent;
Xaa₇ is Lys, Arg, or His, or is absent; and
Xaa₈ is Arg, Lys, or His or is absent.

4-9. (canceled)

10. The polypeptide of claim 1, wherein the fragment of the GDNF precursor protein or the biologically active variant thereof comprises an amino acid sequence of Formula II:
Pro-Pro-Xaa₃-Xaa₄-Pro-Xaa₆-Xaa₇-Xaa₈-Xaa₉-Xaa₁₀-
Xaa₁₁-Xaa₁₂-Xaa₁₃-Xaa₁₄ (II) wherein
Xaa₃ is Glu or Asp;
Xaa₄ is Ala, Gly, Ile, Leu, Met, or Val;
Xaa₆ is Ala, Gly, Ile, Leu, Met, or Val;
Xaa₇ is Glu or Asp;
Xaa₈ is Asp or Glu;
Xaa₉ is Arg, His, or Lys;
Xaa₁₀ is Ser, Asn, Gln, or Thr;
Xaa₁ is Leu, Ala, Gly, Ile, Leu, Met or Val;
Xaa₁₂ is Gly, is any amino acid residue of the D-configuration or is absent;
Xaa₁₃ is Arg, His, or Lys or is absent;
Xaa₁₄ is Arg, His, or Lys or is absent.

11-21. (canceled)

22. The polypeptide of claim 1, wherein the fragment of the GDNF precursor protein or the biologically active variant thereof comprises an amino acid sequence of Formula III:
Xaa₁-Xaa₂-Xaa₃-Xaa₄-Xaa₅-Xaa₆-Xaa₇-Xaa₈-Xaa₉-
Xaa₁₀-Xaa₁₁-Xaa₁₂-Xaa₁₃-Xaa₁₄-Xaa₁₅-Xaa₁₆-Xaa₁₇-
Xaa₁₈-Xaa₁₉-Xaa₂₀-Xaa₂₁-Xaa₂₂ (III),
wherein
Xaa₁ and Xaa₂ are, independently, Arg, Lys, or His;
Xaa₃ is Glu or Asp;
Xaa₄ is Arg, Lys, or His;
Xaa₅ is Asn, Gln, Ser, or Thr;
Xaa₆ is Arg, Lys, or His;
Xaa₇ is Gln, Asn, Ser, or Thr;
Xaa₈, Xaa₉, Xaa₁₀, and Xaa₁₁ are, independently, Ala, Gly, Ile, Leu, Met, or Val;
Xaa₁₂ is Asn, Gln, Ser, or Thr;
Xaa₁₃ is Pro or Ser;
Xaa₁₄ is Glu or Asp;
Xaa₁₅ is Asn, Gln, Ser, or Thr;
Xaa₁₆ is Ser, Asn, Gln, or Thr;
Xaa₁₇ is Lys, Arg, or His;
Xaa₁₈ is Gly, Ala, Ile, Leu, Met, or Val;
Xaa₁₉ is Lys, Arg, or His;
Xaa₂₀ is Gly, is any amino acid residue of the D-configuration or is absent;
Xaa₂₁ and Xaa₂₂ are, independently, Arg, Lys, His, or are absent.

23-39. (canceled)

40. The polypeptide of claim 1, wherein the fragment of the GDNF precursor protein or the biologically active variant thereof is amidated.

41. The polypeptide of claim 1, wherein the fragment of the GDNF precursor protein or the biologically active variant thereof further comprises a detectable marker.

42. The polypeptide of claim 1, wherein the fragment of the GDNF precursor protein has an amino acid sequence naturally expressed in a human cell.

43. The polypeptide of claim 1, wherein the polypeptide is a biologically active variant of the GDNF precursor protein that differs from a wild type fragment of the GDNF precursor protein by virtue of containing one or more conservative amino acid substitutions, with the proviso that at least 50% of the amino acid residues of the variant are identical to residues in the corresponding wild type fragment of the GDNF precursor protein.

44. A pharmaceutically acceptable composition comprising the polypeptide of claim 1.

45. A nucleic acid sequence encoding the polypeptide of claim 1.

46. An expression vector comprising the nucleic acid sequence of claim 45.

47. A host cell comprising the expression vector of claim 46.

48. A method of treating a patient diagnosed as having a neurological disorder, the method comprising administering to the patient the pharmaceutical composition of claim 44.

49-55. (canceled)


SEQUENCE LISTING (67 sequences)

SEQ ID NO: 1 - protein, 8 aa, Homo sapiens
FPLPAGKR

SEQ ID NO: 2 - protein, 14 aa, Homo sapiens
PPEAPAEDRSLGRR

SEQ ID NO: 3 - protein, 22 aa, Homo sapiens
RRERNRQAAAANPENSRGKGRR

SEQ ID NO: 4 - protein, 5 aa, Homo sapiens
FPLPA

SEQ ID NO: 5 - protein, 11 aa, Homo sapiens
PPEAPAEDRSL

SEQ ID NO: 6 - protein, 17 aa, Homo sapiens
ERNRQAAAANPENSRGK

SEQ ID NO: 7 - DNA, 836 bp, Homo sapiens
  CDS (201)...(833)
ccgcctccagcgcgcccttgctgccccgcgcgaccccaggattgcgaactcttgcccctgacctgttgggcggggctccgcgctccagccatcagcccggatgggtctcctggctgggacttggggcacctggagttaatgtccaacctagggtctgcggagacccgatccgaggtgccgccgccggacgggactttaagatgaagttatgggatgtcgtggctgtctgcctggtgctgctccacaccgcgtccgccttcccgctgcccgccggtaagaggcctcccgaggcgcccgccgaagaccgctccctcggccgccgccgcgcgcccttcgcgctgagcagtgactcaaatatgccagaggattatcctgatcagttcgatgatgtcatggattttattcaagccaccattaaaagactgaaaaggtcaccagataaacaaatggcagtgcttcctagaagagagcggaatcggcaggctgcagctgccaacccagagaattccagaggaaaaggtcggagaggccagaggggcaaaaaccggggttgtgtcttaactgcaatacatttaaatgtcactgacttgggtctgggctatgaaaccaaggaggaactgatttttaggtactgcagcggctcttgcgatgcagctgagacaacgtacgacaaaatattgaaaaacttatccagaaatagaaggctggtgagtgacaaagtagggcaggcatgttgcagacccatcgcctttgatgatgacctgtcgtttttagatgataacctggtttaccatattctaagaaagcattccgctaaaaggtgtggatgtatctga

SEQ ID NO: 8 - protein, 211 aa, Homo sapiens
MKLWDVVAVCLVLLHTASAFPLPAGKRPPEAPAEDRSLGRRRAPFALSSDSNMPEDYPDQFDDVMDFIQATIKRLKRSPDKQMAVLPRRERNRQAAAANPENSRGKGRRGQRGKNRGCVLTAIHLNVTDLGLGYETKEELIFRYCSGSCDAAETTYDKILKNLSRNRRLVSDKVGQACCRPIAFDDDLSFLDDNLVYHILRKHSAKRCGCI

SEQ ID NO: 9 - protein, 8 aa, Artificial Sequence
  exemplary motif
XPXPXXXX

SEQ ID NO: 10 - protein, 8 aa, Artificial Sequence
  exemplary motif
FPXPXXXX

SEQ ID NO: 11 - protein, 8 aa, Artificial Sequence
  exemplary motif
XPLPXXXX

SEQ ID NO: 12 - protein, 8 aa, Artificial Sequence
  exemplary motif
FPLPXXXX

SEQ ID NO: 13 - protein, 8 aa, Artificial Sequence
  exemplary motif
XPXPAXXX

SEQ ID NO: 14 - protein, 8 aa, Artificial Sequence
  exemplary motif
FPXPAXXX

SEQ ID NO: 15 - protein, 8 aa, Artificial Sequence
  exemplary motif
FPLPAXXX

SEQ ID NO: 16 - protein, 8 aa, Artificial Sequence
  exemplary motif
XPXPXGXX

SEQ ID NO: 17 - protein, 8 aa, Artificial Sequence
  exemplary motif
FPXPXGXX

SEQ ID NO: 18 - protein, 8 aa, Artificial Sequence
  exemplary motif
FPLPXGXX

SEQ ID NO: 19 - protein, 8 aa, Artificial Sequence
  exemplary motif
FPLPAGXX

SEQ ID NO: 20 - protein, 8 aa, Artificial Sequence
  exemplary motif
XPXPXXKX

SEQ ID NO: 21 - protein, 8 aa, Artificial Sequence
  exemplary motif
FPXPXXKX

SEQ ID NO: 22 - protein, 8 aa, Artificial Sequence
  exemplary motif
FPLPXXKX

SEQ ID NO: 23 - protein, 8 aa, Artificial Sequence
  exemplary motif
FPLPAXKX

SEQ ID NO: 24 - protein, 8 aa, Artificial Sequence
  exemplary motif
FPLPAGKX

SEQ ID NO: 25 - protein, 8 aa, Artificial Sequence
  exemplary motif
XPXPXXXR

SEQ ID NO: 26 - protein, 8 aa, Artificial Sequence
  exemplary motif
FPXPXXXR

SEQ ID NO: 27 - protein, 8 aa, Artificial Sequence
  exemplary motif
FPLPXXXR

SEQ ID NO: 28 - protein, 8 aa, Artificial Sequence
  exemplary motif
FPLPAXXR

SEQ ID NO: 29 - protein, 8 aa, Artificial Sequence
  exemplary motif
FPLPAGXR

SEQ ID NO: 30 - protein, 14 aa, Artificial Sequence
  exemplary motif
PPXXPXXXXXXXXX

SEQ ID NO: 31 - protein, 14 aa, Artificial Sequence
  exemplary motif
PPEXPXXXXXXXXX

SEQ ID NO: 32 - protein, 14 aa, Artificial Sequence
  exemplary motif
PPDXPXXXXXXXXX

SEQ ID NO: 33 - protein, 14 aa, Artificial Sequence
  exemplary motif
PPEXPXEXXXXXXX

SEQ ID NO: 34 - protein, 14 aa, Artificial Sequence
  exemplary motif
PPEXPXDXXXXXXX

SEQ ID NO: 35 - protein, 14 aa, Artificial Sequence
  exemplary motif
PPEXPXEDXXXXXX

SEQ ID NO: 36 - protein, 14 aa, Artificial Sequence
  exemplary motif
PPEXPXEEXXXXXX

SEQ ID NO: 37 - protein, 14 aa, Artificial Sequence
  exemplary motif
PPEXPXDDXXXXXX

SEQ ID NO: 38 - protein, 14 aa, Artificial Sequence
  exemplary motif
PPEXPXDEXXXXXX

SEQ ID NO: 39 - protein, 22 aa, Artificial Sequence
  exemplary motif
XXXXXXXXXXXXXXXXXXXXXX

SEQ ID NO: 40 - protein, 22 aa, Artificial Sequence
  exemplary motif
RRERNRQAAAANPENSKGKGRR

SEQ ID NO: 41 - protein, 22 aa, Artificial Sequence
  exemplary motif
RRERNRQAAAASPENSRGKGRR

SEQ ID NO: 42 - protein, 22 aa, Artificial Sequence
  exemplary motif
KRERNRQAAAANPENSRGKGRR

SEQ ID NO: 43 - protein, 22 aa, Artificial Sequence
  exemplary motif
RKERNRQAAAANPENSRGKGRR

SEQ ID NO: 44 - protein, 22 aa, Artificial Sequence
  exemplary motif
RHERNRQAAAANPENSRGKGRR

SEQ ID NO: 45 - protein, 22 aa, Artificial Sequence
  exemplary motif
HHERNRQAAAANPENSRGKGRR

SEQ ID NO: 46 - protein, 5 aa, Rattus norvegicus
FPLPA

SEQ ID NO: 47 - protein, 8 aa, Rattus norvegicus
FPLPAGKR

SEQ ID NO: 48 - protein, 5 aa, Mus musculus
FPLPA

SEQ ID NO: 49 - protein, 8 aa, Mus musculus
FPLPAGKR

SEQ ID NO: 50 - protein, 14 aa, Rattus norvegicus
LLEAPAEDHSLGHR

SEQ ID NO: 51 - protein, 14 aa, Mus musculus
LLEAPAEDHSLGHR

SEQ ID NO: 52 - protein, 17 aa, Macaca mulata
ERNRQAAAANPENSKGK

SEQ ID NO: 53 - protein, 22 aa, Macaca mulata
RRERNRQAAAANPENSKGKGRR

SEQ ID NO: 54 - protein, 17 aa, Rattus norvegicus
ERNRQAAAASPENSRGK

SEQ ID NO: 55 - protein, 22 aa, Rattus norvegicus
RRERNRQAAAASPENSRGKGRR

SEQ ID NO: 56 - protein, 17 aa, Mus musculus
ERNRQAAAASPENSRGK

SEQ ID NO: 57 - protein, 22 aa, Mus musculus
RRERNRQAAAASPENSRGKGRR

SEQ ID NO: 58 - protein, 17 aa, Rattus sp.
KRNRQAAAASPENSRGK

SEQ ID NO: 59 - protein, 22 aa, Rattus sp.
RRKRNRQAAAASPENSRGKGRR

SEQ ID NO: 60 - protein, 17 aa, Ailuropoda melanoleuca
ERNRQAAAGSPENARGK

SEQ ID NO: 61 - protein, 22 aa, Ailuropoda melanoleuca
RRERNRQAAAGSPENARGKGRR

SEQ ID NO: 62 - protein, 17 aa, Bos taurus
ERHRQGAAASPESARGK

SEQ ID NO: 63 - protein, 22 aa, Bos taurus
RRERHRQGAAASPESARGKGRR

SEQ ID NO: 64 - protein, 17 aa, Gallus gallus
ERNRQSAATNVENSSKK

SEQ ID NO: 65 - protein, 22 aa, Gallus gallus
RRERNRQSAATNVENSSKKGRR

SEQ ID NO: 66 - protein, 17 aa, Nipponia nippon
ERNRQNAATNIENSSKK

SEQ ID NO: 67 - protein, 22 aa, Nipponia nippon
RRERNRQNAATNIENSSKKGRR